(19) United States
(12) Patent Application Publication
(10) Pub. No.: US 2012/0046688 A1
(43) Pub. Date: Feb. 23, 2012

(54) Clip-Based Systems and Methods for Treating Septal Defects

(76) Inventors: Ryan Abbott, San Jose, CA (US); W. Martin Belef, San Jose, CA (US); Dean Carson, Mountain View, CA (US); Peter Fitzgerald, Portola Valley, CA (US); Ronald J. Jabba, Redwood City, CA (US); James Nielsen, San Francisco, CA (US); Peter Thornton, Los Altos, CA (US)

(21) Appl. No.: 13/283,969

(22) Filed: Oct. 28, 2011

Related U.S. Application Data
(63) Continuation of application No. 11/558,312, filed on Nov. 9, 2006.
Application No. 11/558,312 is a continuation of application No. 11/295,338, filed on Dec. 5, 2005, now abandoned.

(51) Int. Cl. A61B 17/08

(52) U.S. Cl. 606/213

(57) ABSTRACT

Systems and methods for treating internal tissue defects, such as septal defects, with clip-based devices are provided. An exemplary clip-based device includes a tubular body having at least a first and a second deflectable member coupled thereto. The first and second members are coupled on opposite ends of the tubular body and configured to deflect between an undeployed configuration and a deployed configuration. In the deployed configuration, each member extends outwardly away from the tubular body in a position configured to abut a tissue surface. The first and second members are preferably configured to maintain a tissue wall therebetween and at least partially close any opening in the tissue wall.

### CROSS-REFERENCE TO RELATED APPLICATIONS

This application is a continuation of U.S. patent application Ser. No. 11/558,312, filed Nov. 9, 2006, which is a continuation of U.S. patent application Ser. No. 11/295,338, filed on Dec. 5, 2005, each of which are fully incorporated herein by reference.

### FIELD OF THE INVENTION

The present invention relates generally to clips for treating internal tissue defects, such as septal defects, and systems and methods for delivering the same.

### BACKGROUND OF THE INVENTION

By nature of their location, the treatment of internal tissue defects is inherently difficult. Access to a defect through invasive surgery introduces a high level of risk that can result in serious complications for the patient. Access to the defect remotely with a catheter or equivalent device is less risky, but treatment of the defect itself is made more difficult given the limited physical abilities of the catheter. The difficulty in accessing and treating tissue defects is compounded when the defect is found in or near a vital organ. For instance, a patent foramen ovale (“PFO”) is a serious septal defect that can occur between the left and right atria of the heart and a patent ductus arteriosus (“PDA”) is an abnormal shunt between the aorta and pulmonary artery.

During development of a fetus in utero, oxygen is transferred from maternal blood to fetal blood through complex interactions between the developing fetal vasculature and the mother's placenta. During this process, blood is not oxygenated within the fetal lungs. In fact, most of the fetus' circulation is shunted away from the lungs through specialized vessels and foramens that are open during fetal life, but typically will close shortly after birth. Occasionally, however, these foramen fail to close and create hemodynamic problems, which, in extreme cases, can prove fatal. During fetal life, an opening called the foramen ovale allows blood to bypass the lungs and pass directly from the right atrium to the left atrium. Thus, blood that is oxygenated via gas exchange with the placenta may travel through the vena cava into the right atrium, through the foramen ovale into the left atrium, and from there into the left ventricle for delivery to the fetal systemic circulation. After birth, with pulmonary circulation established, the increased left atrial blood flow and pressure causes the functional closure of the foramen ovale and, as the heart continues to develop, this closure allows the foramen ovale to grow completely sealed.

In some cases, however, the foramen ovale fails to close entirely. This condition, known as a PFO, can allow blood to continue to shunt between the left and right atria of the heart throughout the adult life of the individual. A PFO can pose serious health risks for the individual, including strokes and migraines. The presence of PFO's have been implicated as a possible contributing factor in the pathogenesis of migraines. Two current hypothesis that link PFO's with migraine include the transit of vasoactive substances or thrombus/emboli from the venous circulation directly into the left atrium without passing through the lungs where they would normally be deactivated or filtered respectively. Other diseases that have been associated with PFO's (and which could benefit from PFO closure) include but are not limited to depression and affective disorders, personality and anxiety disorders, pain, stroke, TIA, dementia, epilepsy, and sleep disorders.

Still other septal defects can occur between the various chambers of the heart, such as atrial-septal defects (ASD's), ventricular-septal defects (VSD's), and the like. To treat these defects as well as PFO's, open heart surgery can be performed to ligate or patch the defect closed. Alternatively, catheter-based procedures have been developed that require introducing umbrella or disc-like devices into the heart. These devices include opposing expandable structures connected by a hub or waist. Generally, in an attempt to close the defect, the device is inserted through the natural opening of the defect and the expandable structures are deployed on either side of the septum to secure the tissue surrounding the defect between the umbrella or disc-like structure.

These devices suffer from numerous shortcomings. For instance, these devices typically involve frame structures that often support membranes, either of which may fail during the life of the patient, thereby introducing the risk that the defect may reopen or that portions of the device could be released within the patient's heart. These devices can fail to form a perfect seal of the septal defect, allowing blood to continue to shunt through the defect. Also, the size and expansive nature of these devices makes safe withdrawal from the patient difficult in instances where withdrawal becomes necessary. The presence of these devices within the heart typically requires the patient to use anti-coagulant drugs for prolonged periods of time, thereby introducing additional health risks to the patient. Furthermore, these devices can come into contact with other portions of the heart tissue and cause undesirable side effects such as an arrhythmia, local tissue damage, and perforation.

Accordingly, improved devices, systems and methods for treating and closing internal tissue defects within the heart are needed.

### SUMMARY

Improved clip-based devices, systems and methods for closing internal tissue defects, such as septal defects and the like, are provided in this section by the way of exemplary embodiments. These embodiments are examples only and are not intended to limit the invention.

In one exemplary embodiment, a medical device for treating internal tissue defects includes a tubular elongate body having an inner lumen, a first member coupled with the tubular body, the first member being biased to deflect outwardly away from the inner lumen into a position configured to abut a first tissue surface, and a second member coupled with the tubular body, the second member being biased to deflect outwardly away from the inner lumen into a position configured to abut a second tissue surface, the first and second members being configured to maintain the first and second tissue surfaces therebetween.

In another exemplary embodiment, a medical device for treating internal tissue defects includes a substantially rigid body comprising a first end portion and a second end portion each located along a first axis of the body, the first and second body portions being flexibly coupled together and separated by a variable distance, a first member having a base coupled with the first end portion, the first member being deflectable between a first orientation and a second orientation, wherein a portion of the first member is offset from the first axis by a greater amount in the first orientation than in the second orientation, and a second member having a base coupled with the second end portion, the second member being deflectable between a first orientation and a second orientation, wherein a portion of the second member is offset from the first axis by a greater amount in the first orientation than in the second orientation.

In one exemplary embodiment of a treatment system for treating a septal defect, the system includes a clip having a substantially rigid body and a plurality of deflectable members coupled with the body, the deflectable members being configured to deflect from an undeployed configuration to a deployed configuration, wherein the clip is deliverable into a septal wall and configured to at least partially close a septal defect in the septal wall with the plurality of deflectable members in the deployed configuration, and an elongate delivery device configured to deliver the clip to the septal wall.

In one exemplary embodiment of a method of treating a septal defect, the method includes delivering a clip having a tubular body into a hole extending through at least a portion of a septal wall, the tubular body comprising a first deflectable member and a second deflectable member, deflecting the first member to a position abutting a first septal tissue surface located on a first side of the septal wall, and deflecting the second member to a position abutting a second tissue surface located on a second side of the septal wall, such that a septal defect tunnel in the septal wall is maintained in an at least partially closed state between the first and second members.

In one exemplary embodiment of a method of manufacturing a medical device configured to treat a septal defect, the method includes forming a clip pattern portion from a tube of a shape memory material, the clip pattern portion comprising a first end portion with a first member coupled thereto, a second end portion with a second member coupled thereto, and a central portion located between the first and second end portions, and treating the clip pattern portion such that the first and second members are biased to deflect outwardly.

In another exemplary embodiment of a method of manufacturing a medical device configured to treat a septal defect, the method includes forming a clip pattern portion from a sheet of a shape memory material, the clip pattern portion comprising a first end portion with a first member coupled thereto, a second end portion with a second member coupled thereto, and a central portion located between the first and second end portions, shaping the clip pattern portion of the sheet into a tubular configuration, configuring the clip pattern portion to retain the tubular configuration, and treating the clip pattern portion such that the first and second members are biased to deflect outwardly.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims. It is also intended that the invention is not limited to require the details of the example embodiments.

### BRIEF DESCRIPTION OF THE FIGURES

The details of the invention, both as to its structure and operation, may be gleaned in part by study of the accompanying figures, in which like reference numerals refer to like parts. The components in the figures are not necessarily to scale, emphasis instead being placed upon illustrating the principles of the invention. Moreover, all illustrations are intended to convey concepts, where relative sizes, shapes and other detailed attributes may be illustrated schematically rather than literally or precisely.

FIG. 1 is a block diagram depicting an exemplary embodiment of a treatment system for treating internal tissue defects.

FIG. 2A is an exterior/interior view depicting an example human heart with a portion of the inferior vena cava and the superior vena cava connected thereto.

FIG. 2B-C are enlarged views of a septal wall taken from FIG. 2A depicting a PFO region.

FIG. 2D is a cross-sectional view depicting a PFO region taken along line 2D-2D of FIGS. 2B-C.

FIG. 3A is a partial cross-sectional view depicting an exemplary embodiment of a clip for treating a PFO in an undeployed configuration.

FIG. 3B is a frontal view depicting an exemplary embodiment of the clip in a deployed configuration.

FIG. 3C is a perspective view depicting an exemplary embodiment of the clip.

FIGS. 4A-D are partial cross-sectional views depicting additional exemplary embodiments of the clip during an exemplary deployment procedure in a heart.

FIG. 4E is a partial cross-sectional view depicting an exemplary embodiment of system 100 using a curved needle to deliver the clip.

FIG. 4F is a frontal view depicting another exemplary embodiment of the clip.

FIG. 4G is a partial cross-sectional view depicting another exemplary embodiment of the clip shown deployed within the septal wall.

FIG. 5A is a perspective view of another exemplary embodiment of the clip in the deployed configuration.

FIG. 5B is a perspective view of another exemplary embodiment of the clip in the undeployed configuration.

FIG. 5C is a frontal view depicting another exemplary embodiment of the clip in the deployed configuration.

FIGS. 5D-E are an end-on views depicting additional exemplary embodiments of the clip.

FIG. 5F is a perspective view depicting another exemplary embodiment of the clip.

FIG. 5G is a partial cross-sectional view depicting the proximal portion of another exemplary embodiment of the clip.

FIG. 5H is a perspective view depicting an exemplary embodiment of a pusher member for use in deploying the clip.

FIGS. 5I-J are perspective views depicting another exemplary embodiment of the pusher member.

FIGS. 6A-C are perspective views depicting additional exemplary embodiments of the clip implanted within the septal wall.

FIG. 7A is a perspective view depicting an exemplary embodiment of the clip formed from a NITINOL sheet for use in an exemplary fabrication process.

FIG. 7B is a perspective view depicting an exemplary embodiment of the clip during an exemplary fabrication process.

FIG. 7C is a frontal view depicting another exemplary embodiment of the clip.

FIG. 7D is a perspective view depicting another exemplary embodiment of the clip.

FIG. 7E is a frontal view depicting the central portion of another exemplary embodiment of the clip during an exemplary fabrication process.

FIG. 7F is a perspective view depicting another exemplary embodiment of the clip formed from a NITINOL sheet for use in an exemplary fabrication process.

FIG. 7G is a frontal view depicting another exemplary embodiment of the clip.

FIGS. 7H-I are frontal views depicting the central portion of additional exemplary embodiments of the clip during an exemplary fabrication process.

FIGS. 7J-K are frontal views depicting additional exemplary embodiments of the clip.

FIG. 8A is an end-on view depicting an exemplary embodiment of a left atrial member in the deployed configuration.

FIG. 8B is a partial cross-sectional view depicting another exemplary embodiment of the left atrial member in the undeployed configuration.

FIG. 8C is an end-on view depicting another exemplary embodiment of the clip.

FIG. 8D is a frontal view depicting another exemplary embodiment of the clip.

FIGS. 9A-B are perspective views depicting additional exemplary embodiments of the clip in a deployed state.

FIGS. 10-11 are perspective views depicting additional exemplary embodiments of the clip.

FIGS. 12A-D are perspective views depicting additional exemplary embodiments of a portion of the left atrial member.

FIG. 12E is an end-on view of another exemplary embodiment of the clip.

FIGS. 13A-B are frontal views depicting additional exemplary embodiments of the clip.

FIGS. 13C-D are perspective views depicting additional exemplary embodiments of an end portion of the clip.

FIGS. 13E-F are perspective views of additional exemplary embodiments of the end portion of the clip.

FIG. 14A is a perspective view depicting another exemplary embodiment of the clip in the undeployed configuration.

FIG. 14B is a frontal view depicting another exemplary embodiment of the clip in the deployed configuration.

FIGS. 15A-D are end-on views depicting additional exemplary embodiments of the clip.

FIGS. 16A-D are perspective views depicting additional exemplary embodiments of the end portion of the clip.

FIGS. 16E-F are perspective views depicting additional exemplary embodiments of an end portion of the clip.

FIG. 17A is a perspective view depicting another exemplary embodiment of the clip.

FIGS. 17B-C are enlarged perspective views depicting additional exemplary embodiments of the end portion of the clip.

FIG. 17D is an end-on view depicting another exemplary embodiment of the clip.

FIGS. 17E-F are enlarged perspective views depicting exemplary embodiments of an end tip 402 coupled with the clip body.

FIGS. 17G-H are end-on views depicting additional exemplary embodiments of the clip.

FIGS. 17I-J are frontal views depicting additional exemplary embodiments of the clip.

FIGS. 18A-B are frontal views depicting additional exemplary embodiments of the clip.

FIGS. 19A-B are partial cross-sectional views depicting additional exemplary embodiments of the clip during deployment into a septal wall.

FIGS. 20A-B are frontal views depicting additional exemplary embodiments of the clip.

FIG. 21A is a cross-sectional view depicting another exemplary embodiment of the clip.

FIG. 21B is a frontal view depicting another exemplary embodiment of the clip.

FIG. 22A is a frontal view depicting another exemplary embodiment of the clip.

FIG. 22B is a partial cross-sectional view depicting another exemplary embodiment of the clip.

FIG. 23A is a frontal view depicting another exemplary embodiment of the clip.

FIG. 23B is a cross-sectional view depicting another exemplary embodiment of the clip.

FIG. 23C is a frontal view depicting another exemplary embodiment of the clip.

FIGS. 24A-B are frontal and end-on views, respectively, depicting additional exemplary embodiments of the clip.

FIG. 24C is a cross-sectional view depicting another exemplary embodiment of the clip.

FIG. 24D is a frontal view depicting another exemplary embodiment of the clip.

FIG. 25A is a perspective view depicting another exemplary embodiment of the clip.

FIG. 25B is a partial cross-sectional view depicting another exemplary embodiment of the clip.

FIGS. 25C-D are perspective views depicting additional exemplary embodiments of the clip.

FIG. 25E is a frontal view depicting another exemplary embodiment of the clip.

FIG. 25F is a partial cross-sectional view of another exemplary embodiment taken along line 25F-25F of FIG. 25E.

FIG. 25G is a partial cross-sectional view of another exemplary embodiment taken along line 25G-25G of FIG. 25F.

FIGS. 26A-D are partial cross-sectional views depicting additional exemplary embodiments of the clip during an exemplary retrieval process.

FIGS. 26E-F are perspective views depicting additional exemplary embodiments of the clip.

FIG. 26G is a frontal view depicting another exemplary embodiment of the clip.

FIG. 27A is a partial cross-sectional view depicting an exemplary embodiment of the treatment system.

FIG. 27B is a lateral view depicting an exemplary embodiment of the pusher member from a perspective rotated 90 degrees from that of the depiction in FIG. 27A.

FIG. 27C is a partial cross-sectional view depicting another exemplary embodiment of the treatment system.

FIGS. 28A-B are partial cross-sectional views depicting additional exemplary embodiments of the treatment system.

FIG. 29A is a frontal view depicting another exemplary embodiment of the clip in an uncoupled state.

FIG. 29B is a cross-sectional view depicting another exemplary embodiment of the clip in a coupled, deployed configuration.

FIG. 30A is a frontal view depicting another exemplary embodiment of the clip in an uncoupled state.

FIG. 30B is a cross-sectional view depicting another exemplary embodiment of the clip in a coupled state.

FIG. 31A is a perspective view of another exemplary embodiment of the clip.

FIGS. 31B-C are cross sectional views of another exemplary embodiment of the clip taken along line 31B-31B of FIG. 31A.

FIG. 32A is a frontal view depicting another exemplary embodiment of the clip.

FIG. 32B is a partial cross-sectional view depicting another exemplary embodiment of the clip implanted within a septal wall.

### DETAILED DESCRIPTION

Deformable clip-type devices for treating internal tissue defects are described herein, along with systems for delivery of those devices as well as methods for using the same. For ease of discussion, these devices, systems and methods will be described with reference to treatment of a PFO. However, it should be understood that these devices, systems and methods can be used in treatment of any type of septal defect including ASD's, VSD's and the like, as well as PDA's, pulmonary shunts or other structural cardiac or vascular defects or non-vascular defects, and also any other tissue defect including non-septal tissue defects.

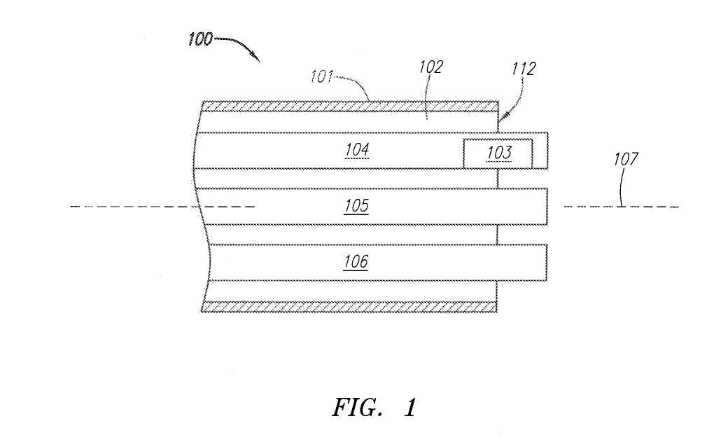

FIG. 1 is a block diagram depicting a distal portion of an exemplary embodiment of a septal defect treatment system 100 configured to treat and preferably close a PFO. In this embodiment, treatment system 100 includes an elongate body member 101 configured for insertion into the vasculature of a patient (human or animal) having a septal defect. Body member 101 has a longitudinal axis 107, a distal end 112 and can include one or more lumens 102, each of which can be configured for achieving multiple functions. Preferably, treatment system 100 includes an implantable device 103 configured to facilitate partial or entire closure of a septal defect.

Implantable device 103 is preferably configured in a tubular clip-like manner and, to facilitate this description, will be referred to herein as clip 103. Treatment system 100 can include a flexible elongate delivery device 104 configured to house and deliver clip 103. Clip 103 can be deformable (i.e., the shape can be altered or changed by pressure, stress or pre-existing bias), deflectable or shape changeable between a deployed configuration and an undeployed, or housed, configuration. To minimize the radial cross-sectional width of body member 101 and aid in deployment, the lateral cross-sectional profile of clip 103 in the undeployed configuration is preferably smaller than the lateral cross-sectional profile of clip 103 in the deployed configuration. This allows clip 103 to be more compactly housed within delivery device 104 and more easily delivered through or into the septal wall.

Treatment system 100 can also optionally include a stabilization device 105 for stabilization of body member 101 during delivery of clip 103 and a positioning device 106 for facilitating the positioning or the centering of delivery device 104 for delivery. Although shown here as four separate components, any combination of body member 101, delivery device 104, stabilization device 105 and centering device 106 can be integrated together to reduce the number of components to three, two or one total components in treatment system 100. A user can manipulate delivery device 104, stabilization device 105 and centering device 106 at the proximal end of body member 101 (not shown). The use of a similar treatment systems 100, also having body members 101, delivery devices 104, stabilization devices 105 and centering devices 106, are described in detail in co-pending U.S. patent application Ser. Nos. 11/175,814, filed Jul. 5, 2005 and entitled “Systems and Methods for Treating Septal Defects,” and 11/218,794, filed Sep. 1, 2005 and entitled “Suture-based Systems and Methods for Treating Septal Defects,” both of which are fully incorporated by reference herein. Although these applications are directed mainly to the delivery of coil-like and suture-like devices, respectively, many of the delivery methods and systems that are described are equally applicable to clip 103.

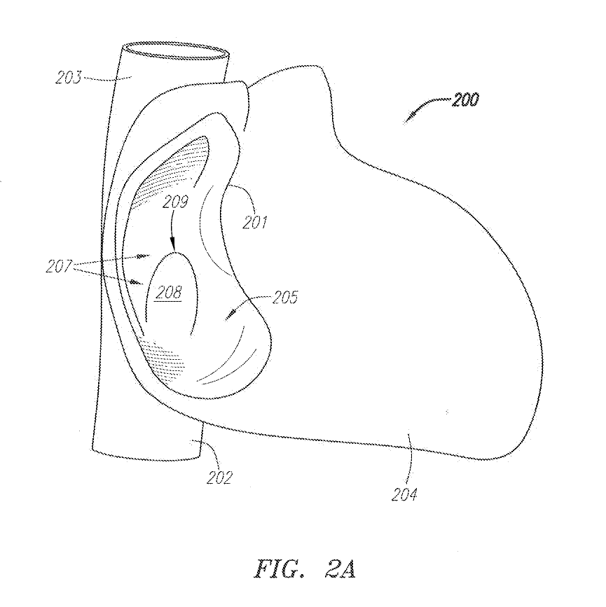

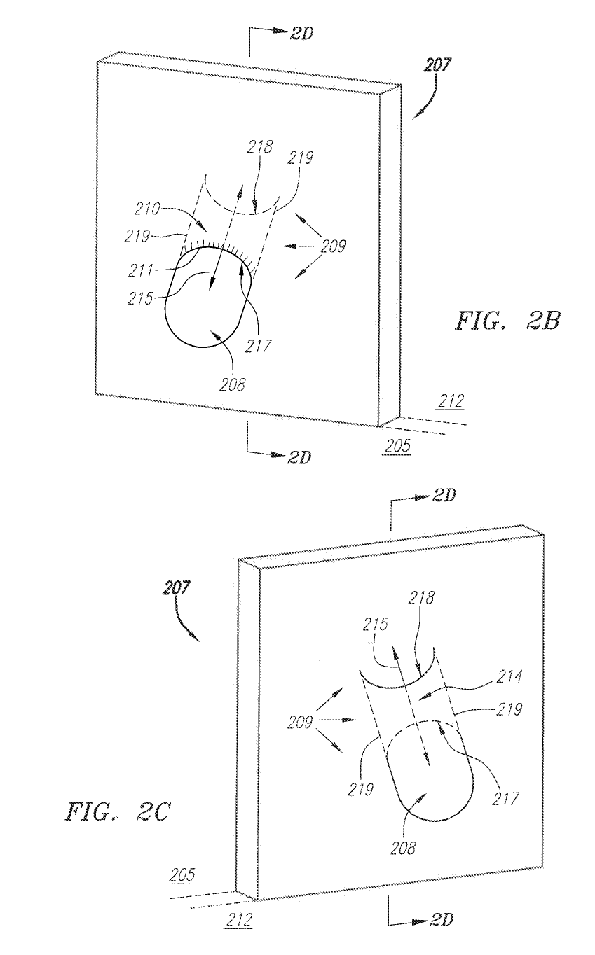

To better understand the many alternative embodiments of treatment system 100, the anatomical structure of an example human heart having a PFO will be described in brief. FIG. 2A is an exterior/interior view depicting an example human heart 200 with a portion of the inferior vena cava 202 and the superior vena cava 203 connected thereto. Outer tissue surface 204 of heart 200 is shown along with the interior of right atrium 205 via cutaway portion 201. Depicted within right atrium 205 is septal wall 207, which is placed between right atrium 205 and the left atrium located on the opposite side (not shown). Also depicted is fossa ovalis 208, which is a region of septal wall 207 having tissue that is relatively thinner than the surrounding tissue. PFO region 209 is located beyond the upper portion of the fossa ovalis 208.

FIG. 2B is an enlarged view of septal wall 207 depicting PFO region 209 in more detail as viewed from right atrium 205. PFO region 209 includes septum secundum 210, which is a first flap-like portion of septal wall 207. The edge of this flap above fossa ovalis 208 is referred to as the limbus 211. FIG. 2C is also an enlarged view of septal wall 207, instead depicting septal wall 207 as viewed from left atrium 212. Here, PFO region 209 is seen to include septum primum 214, which is a second flap-like portion of septal wall 207. Septum primum 214 and septum secundum 210 partially overlap each other and define a tunnel-like opening 215 between sidewalls 219 (indicated as dashed lines in FIGS. 2B-C) that can allow blood to shunt between right atrium 205 and left atrium 212 and is commonly referred to as a PFO.

FIG. 2D is a cross-sectional view depicting an example PFO region 209 taken along line 2D-2D of FIGS. 2B-C. Here, it can be seen that septum secundum 210 is thicker than septum primum 214. Typically, the blood pressure within left atrium 212 is higher than that within right atrium 205 and tunnel 215 remains sealed. However, under some circumstances conditions can occur when the blood pressure within right atrium 205 becomes higher than the blood pressure within left atrium 212 and blood shunts from right atrium 205 to left atrium 212 (e.g., a valsava condition). Because most typical shunts occur in this manner and for purposes of facilitating the discussion herein, region 217 in FIG. 2D will be referred to as PFO entrance 217, and region 218 will be referred to as PFO exit 218.

Many different variations of PFO's can occur. For instance, thickness 220 of septum primum 214, thickness 221 of septum secundum 210, overlap distance 222 and the flexibility and distensibility of both septum primum 214 and septum secundum 210 can all vary. In FIGS. 2B-C, PFO entrance 217 and PFO exit 218 are depicted as being relatively the same size with the width of tunnel 215, or the distance between sidewalls 219, remaining relatively constant. However, in some cases PFO entrance 217 can be larger than PFO exit 218, resulting in an tunnel 215 that converges as blood passes through. Conversely, PFO entrance 217 can be smaller than PFO exit 218, resulting in an opening that diverges as blood passes through. Furthermore, multiple PFO exits 218 can be present, with one or more individual tunnels 215 therebetween. Also, in FIGS. 2B-D, both septum primum 214 and septum secundum 210 are depicted as relatively planar tissue flaps, but in some cases one or both of septum primum 214 and septum secundum 210 can have folded, non-planar, highly irregular shapes.

As will be described in more detail below, treatment of a PFO preferably includes inserting treatment system 100 into the vasculature of a patient and advancing body member 101 through the vasculature to inferior vena cava 202 (e.g., over a guidewire), from which access to right atrium 205 can be obtained. Once properly positioned within right atrium 205, delivery device 104 can be used to deliver one or more clips 103 to PFO region 209, preferably by inserting each clip 103 through septum secundum 210 and primum 214 such that it lies transverse to tunnel 215 to at least partially close tunnel 215. Thus, the use of clip-based devices, systems and methods for treating PFO's allows direct closure of PFO tunnel 215, as opposed to occlusive-type devices that merely block PFO entrance 217 and exit 218 without directly closing tunnel 215.

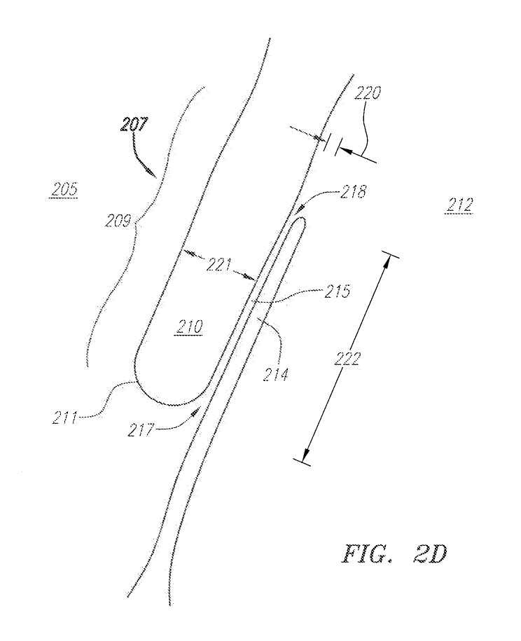

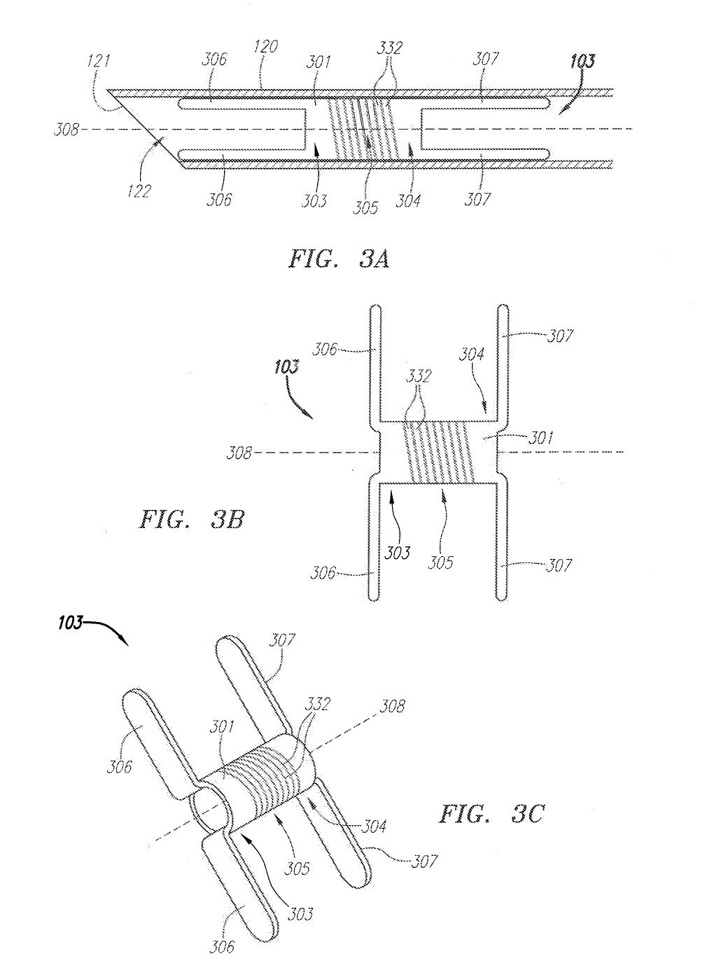

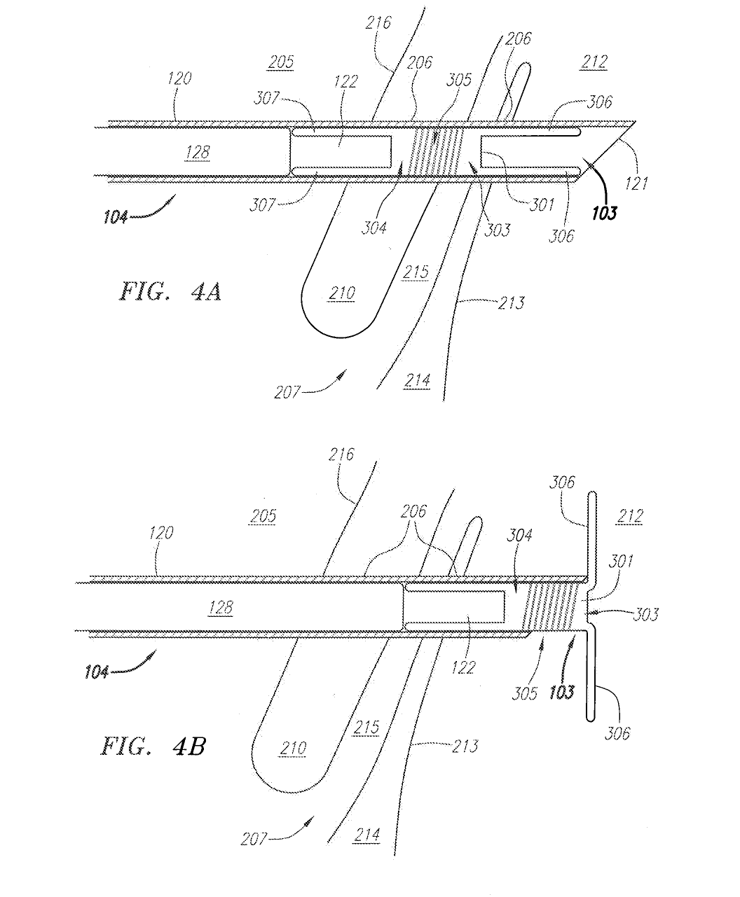

Clip 103 can be configured in numerous different variations. FIGS. 3A-C depict one exemplary embodiment of clip 103. Preferably, clip 103 includes a body 301 having a first, or distal, end portion 303, a second, or proximal, end portion 304 and a central portion 305 located therebetween. Coupled with the first and second end portions 303 and 304 are deflectable (i.e., bendable, shiftable, twistable or turnable) members 306 and 307, respectively, which are configured to abut septal tissue. In this embodiment, clip 103 includes two members 306 and two members 307; however, any number of one or more members 306 can be used with any number of one or more members 307. Deflectable members 306 and 307 are preferably biased to deflect from an undeployed configuration, for facilitating delivery of clip 103, to a deployed configuration, for treating a PFO.

FIG. 3A is a partial cross-sectional view depicting clip 103 in the undeployed configuration with members 306 and 307 oriented generally along a main axis 308 of body 301. Here, clip 103 is shown housed within a cross-section of an elongate tubular member 120 having a substantially sharp distal end 121 and an inner lumen 122. Elongate member 120 restrains deflectable members 306 and 307 from deflection into the deployed state. Although not limited to such, member 120 will be referred to as needle 120 for purposes of facilitating the description herein. FIGS. 3B-C are frontal and perspective views, respectively, depicting clip 103 in the deployed configuration after delivery from within needle 120. Here, members 306 and 307 are deflected outwards such that each member 306 and 307 has a greater offset from main axis 308 than in the undeployed state. When deployed in this configuration, clip 103 preferably holds septum primum 214 and septum secundum 210 together to close PFO tunnel 215.

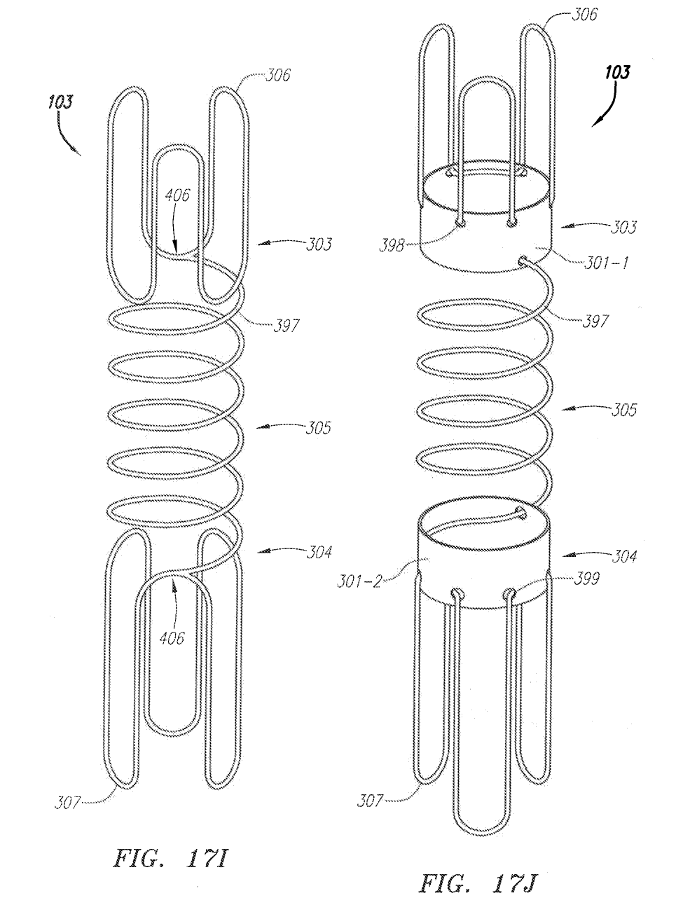

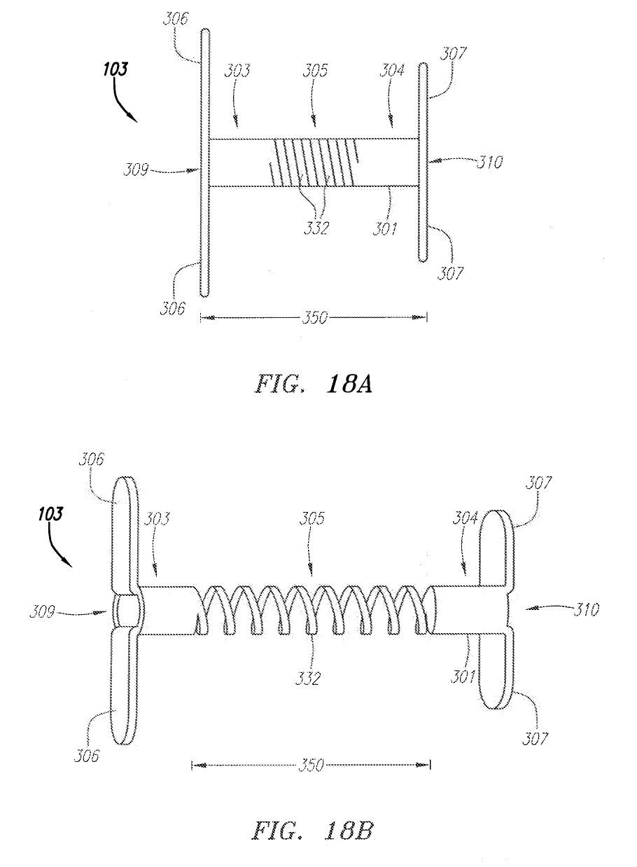

Central portion 305 of clip 103 can be optionally configured to expand and compress to facilitate closure of the PFO. In this embodiment, central portion 305 is configured like a spring with multiple compressive segments 332. The operation of compressible/expandable central portions 305 will be discussed in more detail with reference to FIGS. 18A-24D below. It should be noted that any portion of clip 103 can be made compressible/expandable, not limited solely to central portion 305. For instance, clip 103 can have one or more compressible/expandable end portions 303 and 304 with a rigid central portion 305.

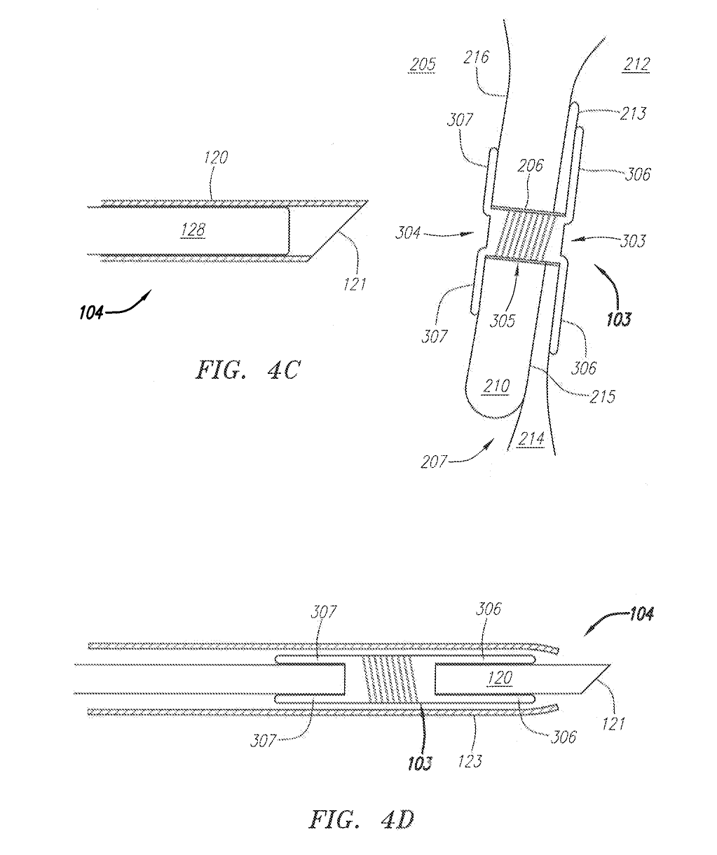

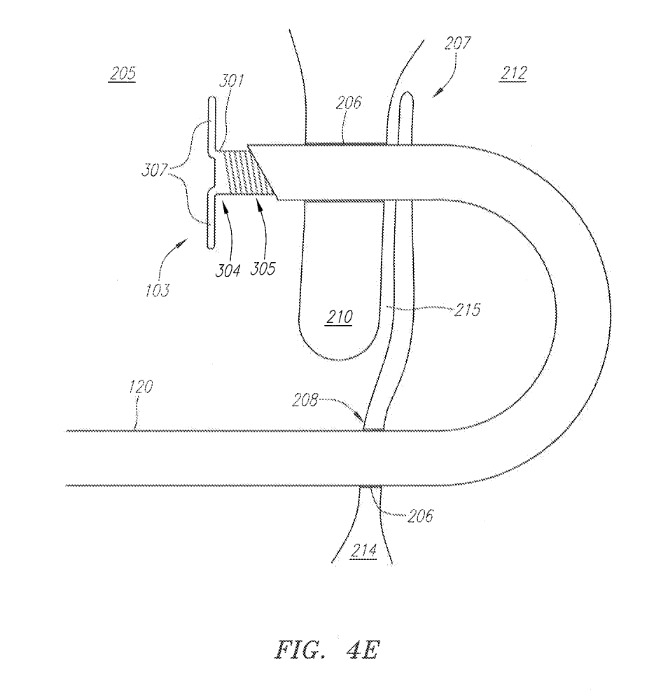

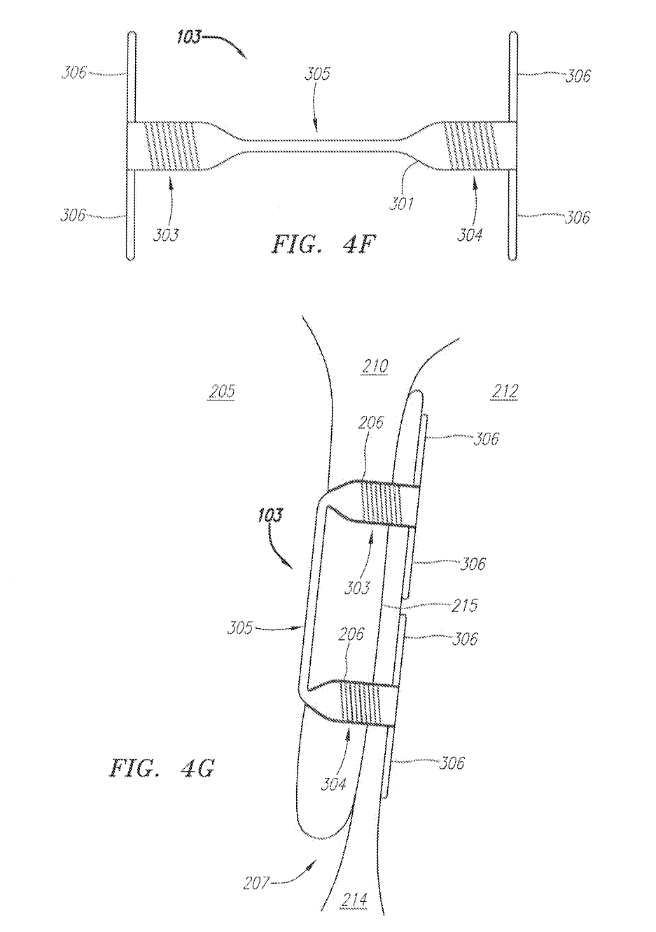

FIGS. 4A-D are partial cross-sectional views depicting the embodiment of clip 103 described with respect to FIGS. 3A-B during an exemplary deployment procedure in heart 200. In this embodiment, needle 120 is preferably positioned adjacent to septal wall 207. Needle 120 is then used to penetrate septal wall 207 by continually advancing needle 120 through septal wall 207 until distal end 121 is exposed within left atrium 212 as depicted in FIG. 4A. This creates an opening 206 through both septum secundum 210 and septum primum 214.

An elongate pusher member 128 is preferably used to deliver clip 103 through opening 206 into left atrium 212. Pusher member 128, which can be slidably disposed within lumen 122, is advanced distally against clip 103 to slide clip 103 in a distal direction until first end portion 303 is exposed from within needle member 120. Once exposed, members 306 are free to deflect towards their biased deployed configuration as depicted in FIG. 4B. Needle member 120 can then be retracted proximally back through septal wall 207. Clip 103 can then be fully deployed from within lumen 122 through continued use of pusher member 120, or by allowing members 306 to “catch” surface 213 and drag clip 103 from lumen 122, or in any other manner desired. Once clip 103 is fully exposed from within needle 120, members 307 are free to deflect towards their biased deployed state as depicted in FIG. 4C.

When fully deployed, clip 103 acts to restrain septum primum 214 and septum secundum 210 from moving apart from one another, reducing the amount of open space within tunnel 215 and preferably closing tunnel 215 altogether. Preferably, members 306 and 307 apply an relatively even or uniform amount of force across septum primum 214 and secundum 210, respectively. The application of an even amount of force acts to flatten and hold primum 214 against secundum 210 to avoid the creation of residual shunts that could occur of primum 214 or secundum 210 bunches up underneath members 306 or 307, respectively.

In this example, deflectable members 306 are deployed in left atrium (LA) 212 and deflectable members 307 are deployed in right atrium (RA) 205. Although not limited to such, in order to facilitate the description herein, deflectable members 306 and 307 will be referred to as LA members 306 and RA members 307, respectively.

As mentioned above, central portion 305 of body 301 is preferably configured to be expandable and compressible to facilitate closure of tunnel 215. In this embodiment, central portion 305 is configured to be an elastic, spring-like portion of body 301. Central portion 305 is preferably biased towards a fully compressed state to effectuate the maximum closure force onto septal wall 207 and tunnel 215. Central portion 305 can expand to accommodate varying thickness of septal wall 207, i.e., in the event that septal wall 207 is thicker than the length of body 301 between LA members 306 and RA members 307.

In the method described above with respect to FIGS. 4A-C, needle 120 is used to house clip 103 prior to deployment. However, clip 103 can be housed in any portion of treatment system 100 as desired. For instance, an outer elongate tubular member, or sheath 123, can be configured to slidably receive needle 120, which in turn can be tubular or solid like a trocar. Clip 103 can reside over top of needle 120 and be housed within sheath 123, as depicted in the partial cross-sectional view of FIG. 4D. In this case, deployment of members 306 and 307 can occur by retracting sheath 123 proximally with respect to needle 120 to expose members 306 and 307 and allow them to catch onto the desired septal surface.

Before puncturing septal wall 207, needle 120 is first properly oriented with respect to septal wall 207. In the example described with respect to FIGS. 4A-C, needle 120 is preferably oriented to be generally perpendicular to septum secundum surface 216 (i.e., oriented generally normal to surface 216). With certain manners of delivery, for instance, if a catheter is used to advance clip 103 into heart 200, treatment system 100 is preferably configured to properly orient needle 120 with respect to septal wall 207. One such configuration is described in further detail in the incorporated co-pending U.S. patent application Ser. No. 11/175,814, filed Jul. 5, 2005 and entitled “Systems and Methods for Treating Septal Defects.” Although the off-axis delivery systems and methods are described primarily with respect to coil-like implantable treatment devices, many of these systems and methods are equally applicable to the clip-like implants 103 described herein.

In the embodiment described with respect to FIGS. 4A-C, clip 103 is delivered from right atrium 205 into left atrium 212. Clip 103 can also be delivered in the opposite direction as well. For instance, device 101 can be routed directly into left atrium 212 and used to deliver clip 103 into right atrium 205. Alternatively, device 101 can be routed into right atrium 205 and a curved needle can be used to puncture septal wall 207 (e.g., fossa ovalis 208) to gain access to left atrium 212. The curved needle 120 can then be routed into left atrium 212 and used to puncture septal wall 207 a second time from left atrium 212 into right atrium 205, creating a second opening into which clip 103 can be deployed.

FIG. 4E is a partial cross-sectional view depicting an exemplary embodiment of system 100 using curved needle 120 to deliver clip 103 from left atrium 212 into right atrium 205. Here, clip 103 is shown in the middle of the deployment process as clip 103 is being advanced from within needle 120 and RA members 307 have deployed within right atrium 205. To complete the deployment, needle 120 is then retracted back through septal wall 207 to fully deploy clip 103, at which point needle 120 can be retracted from left atrium 212 back into right atrium 205. Curved needles and their use are described in further detail in co-pending U.S. patent application Ser. No. 11/218,794, entitled “Suture-based Systems and Methods for Treating Septal Defects.”

Clip 103 can also be delivered through multiple openings 206 in septal wall 207. FIG. 4F is a frontal view depicting an exemplary embodiment of clip 103 in the deployed configuration for delivery through two openings 206. Here, end portions 303 and 304 are configured to be compressible and expandable and central portion 305 is configured to be relatively more narrow and rigid. In this embodiment, both end portions 303 and 304 are configured to be deployed within left atrium 212 and at least part of central portion 305 is configured to reside within right atrium 205. Because both end portions 303 and 304 are configured to reside within left atrium 212, each portion 303 and 304 is coupled with LA members 306 and no RA members 307 are necessary (although the can be used in conjunction with central portion 305 if desired). FIG. 4G is a partial cross-sectional view depicting this embodiment of clip 103 deployed within septal wall 207. The separate openings 206 can be created using a dual-needle such as that described in co-pending U.S. patent application Ser. No. 11/218,794, entitled “Suture-based Systems and Methods for Treating Septal Defects.”

Clip 103 is distinguishable from other septal closure devices such as sutures and suture-based devices. Sutures typically have thread-like, wire-like or filament-like bodies that are easily manipulated and flexible. Also, sutures are bendable and deformable and typically cannot retain any particular layout or shape. Clip 103, on the other hand, preferably has a more robust substantially rigid body 301 that can resist deformation yet at the same time adjust to the contours of the surrounding septal wall 207, in part through the presence of the compressible/expandable central portion 305. Because clip 103 preferably uses deflectable members 306 and 307 to clamp septum primum 214 and secundum 210 together (in addition to central compressive portion 305), the presence of substantially rigid end portions 303 and 304 onto which members 306 and 307 rely to generate sufficient leverage to close PFO tunnel 215 can be a useful characteristic. Also, the substantially rigid body 301 of clip 103 can be made rigid enough to maintain the orientation of LA members 306 with respect to RA members 307, i.e., to resist twisting about main axis 308, whereas a suture is incapable of achieving the same degree of orientational control.

These differences are in addition to the clear structural and operational differences that also exist between the suture/suture-based devices and clip 103. Typical sutures require multi-piece construction, with one or more parts for the suture locking device and/or anchors. Suture thread materials are typically not visible under fluoroscopic imaging. Sutures threads are prone to abrasion, whereas clips 103 fabricated from NITINOL or stainless steel are not. Typical sutures cannot exert continuous compressive force against the septal wall when shifts in the tissue or suture placement occur after deployment. Sutures also require the physician or user to control the closure force of the suture, whereas clip 103 is self-adjusting. Clip 103 can be deployed with a simple pushing motion alone, if desired, whereas the thread-like construction of sutures makes deployment more complex. The use of a suture to close a PFO can cause PFO tunnel 215 to bunch up and create residual shunts. Clip 103 preferably applies an even closure force across both septum primum 214 and secundum 210 that prevents the creation of residual shunts. Also, clip 103 can be deployed via creation of a single opening 215 in septal wall 207. Most typical sutures require at least two punctures for deployment, and therefore risk additional bleeding and tissue damage during the deployment procedure. It should be noted that this list is not exhaustive and only points out some of the many differences that exist between sutures and clip 103.

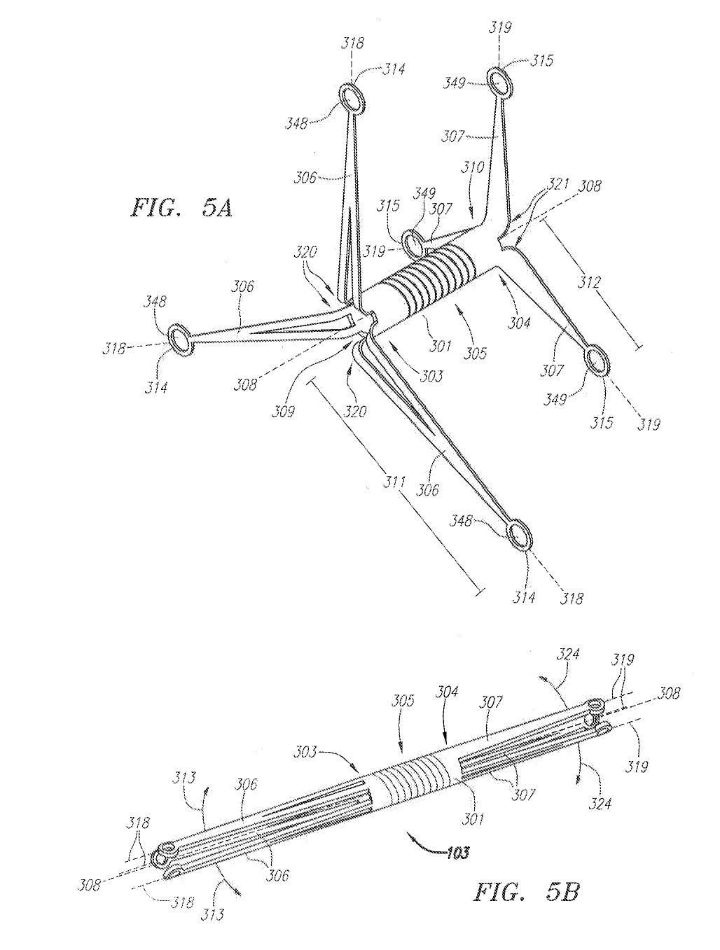

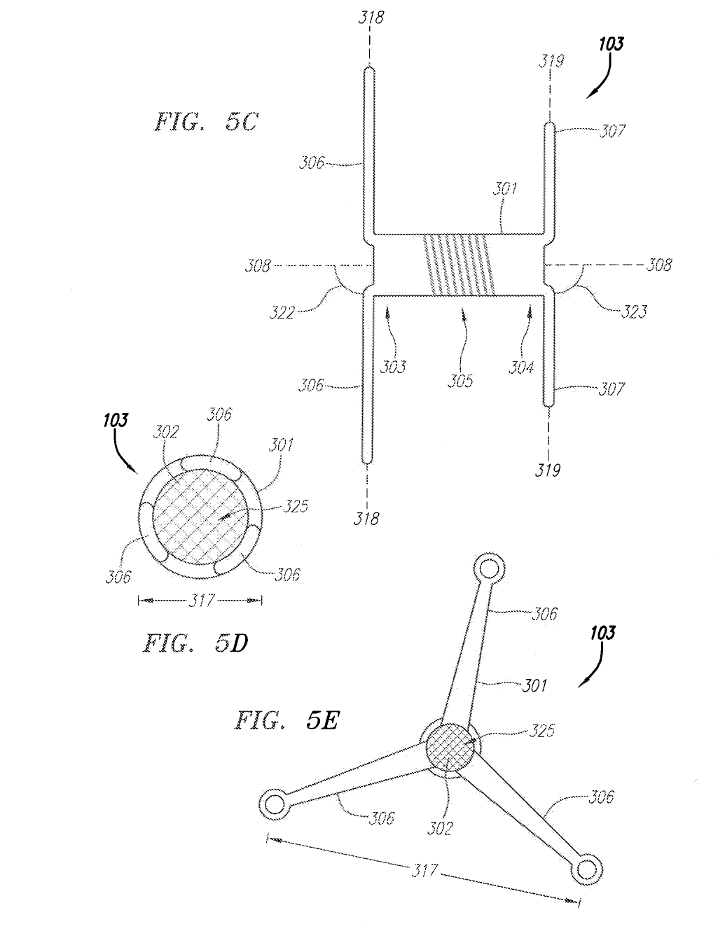

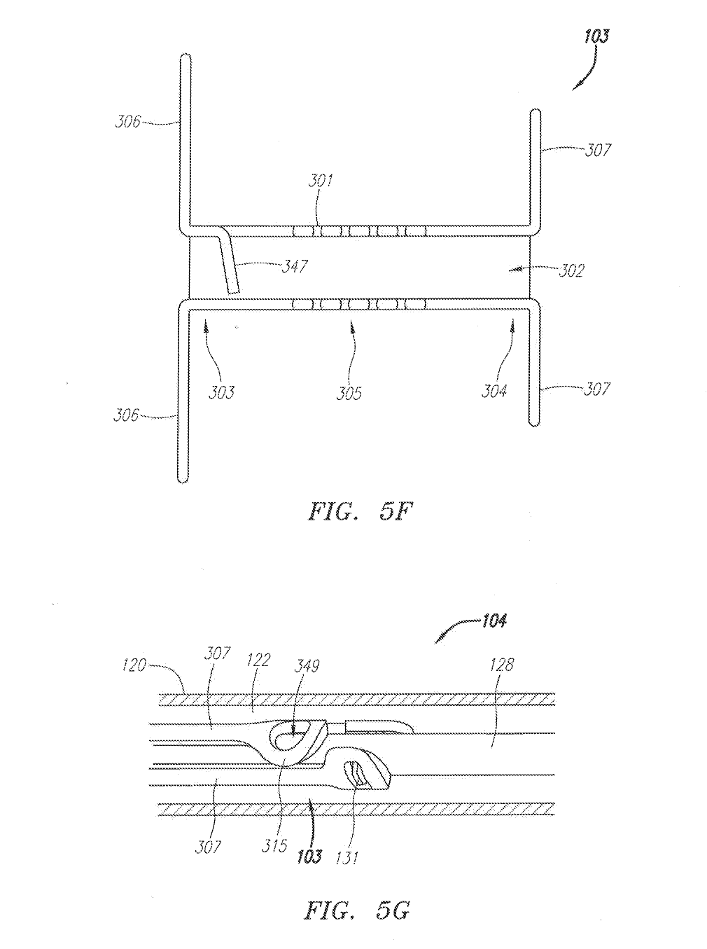

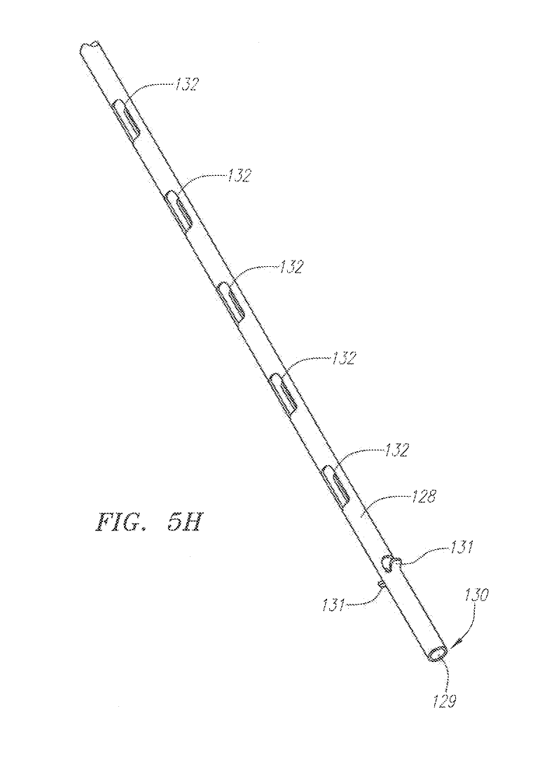

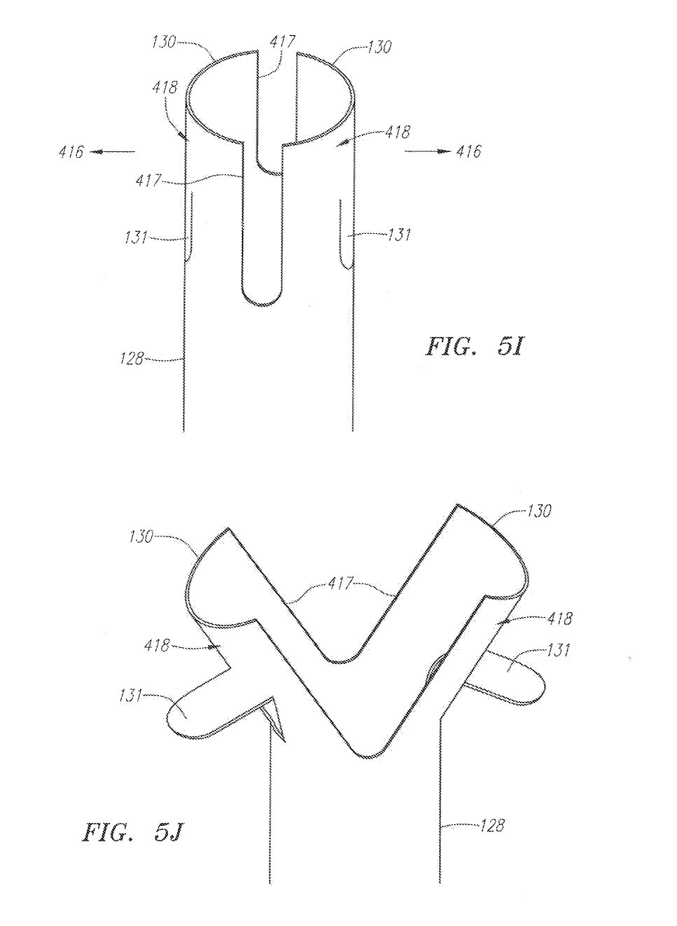

FIGS. 5A-H depict additional exemplary embodiments of clip 103. FIG. 5A is a perspective view of one exemplary embodiment in the deployed configuration. Here, clip 103 includes three LA members 306 and three RA members 307. LA members 306 are coupled to distal end 309 of distal end portion 303 and RA members 307 are coupled to proximal end 310 of proximal end portion 304. In this embodiment, length 311 of each LA member 306 is greater than length 312 of each RA member 307 in part to provide greater surface area coverage over septum primum 214. LA members 306 and RA members 307 have end tips 314 and 315, respectively, that are preferably atraumatic. Here, tips 314 and 315 are annular for added strength and include inner apertures 348 and 349, respectively Inner apertures 348 and 349 allow tissue to mechanically anchor to implant 103 in order to reduce chronic abrasion and potential tissue perforation risks. Although not shown, the atraumatic characteristics of end tips 314 and 315 can be improved by deflecting them away from any adjacent tissue surface. Also, radio opaque markers (e.g., tantalum) can be placed within apertures 348 and 349 to increase the visibility of clip 103 in X-ray imaging. A retrieval tether 316, which will be discussed in more detail below, can also be passed through one or more of inner apertures 348 or 349, if desired.

FIG. 5B is a perspective view of this embodiment of clip 103 in the undeployed configuration. Here, each member 306 and 307 is oriented generally along main axis 308 of body 301. Arrows 313 and 324 indicate the direction in which each LA and RA member 306 and 307, respectively, is biased to deflect. In the undeployed configuration, the entire body 301 of clip 103, including members 306 and 307, has a generally elongate shape, in this case being describable as rod-like or cylindrical.

As shown in FIGS. 5A-B, each LA and RA member 306 and 307 can be described as having a longitudinal axis 318 and 319, respectively. LA longitudinal axis 318 extends from a base portion 320 of each LA member 306 to end tip 314. Likewise, RA longitudinal axis 319 extends from a base portion 321 of each RA member 307 to end tip 315. In the undeployed configuration, these longitudinal axes 318 and 319 are oriented generally along main axis 308, although not necessarily parallel with main axis 308. In the deployed configuration, each longitudinal axis 318 and 319 is offset from main axis 308 by a relatively greater amount than in the undeployed configuration. Viewed differently, longitudinal axes 318 and 319 can be described as being relatively less parallel to main axis 308 in the deployed configuration than in the undeployed configuration. It should be noted that LA and RA members 306 and 307 are not required to be straight in order to have a longitudinal axis 318 and 319, respectively.

FIG. 5C is a frontal view depicting this embodiment of clip 103 in the deployed configuration. Here, each LA and RA member 306 and 307 is offset from main axis 308 by a deflection angle 322 and 323, respectively. Deflection angles 322 and 323 are relatively greater in the deployed configuration than in the undeployed configuration. Here, deflection angles 322 and 323 are all approximately 90 degrees in the deployed configuration, although any deflection angles 322 and 323 can be used. Also, longitudinal axes 318 and 319 are substantially perpendicular to main axis 308 in this deployed configuration (although axes 318 and 319 do not necessarily intersect main axis 308). Although not shown, deflection angles 322 and 323 are approximately zero degrees in the exemplary undeployed configuration of FIG. 5B.

FIGS. 5D-E are end-on views of another embodiment of clip 103 in the undeployed and deployed configurations, respectively. From these views it can be seen that clip 103 has a significantly smaller lateral profile in the undeployed configuration than in the deployed configuration. Width 317 of clip 103 is much greater in the deployed configuration than in the undeployed configuration. This allows clip 103 to be delivered from within a narrow, low profile device, such as needle 120, which can be easily advanced through the patient's confined vasculature into proximity with septal wall 207. This also allows creation of a narrow, low profile puncture, such as manmade opening 206, which can heal in a relatively quick manner with a lesser risk of blood shunting through the puncture. The ability of clip 103 to deflect or expand into a wider deployed configuration allows clip 103 to effectuate closing of PFO tunnel 215 over a wider surface area of septal wall 207.

As can be seen in the embodiment depicted in FIGS. 5D-F, body 301 has an inner lumen 302 which is preferably substantially blocked to prevent significant amounts of blood from shunting between the left and right atria through inner lumen 302. In the embodiments depicted in FIGS. 5D-E, inner lumen 302 is filled with a blocking material 325. Here, blocking material 325 is a multitude of polyester fibers attached to the inner surface of inner lumen 302. Any type of blocking material 325 can be used as desired. In other exemplary embodiments, a physical plug can be placed in lumen 302 to prevent shunting, or body 301 can be solid with no inner lumen 302 to prevent shunting and the like. FIG. 5F is a perspective view depicting another exemplary embodiment of clip 103 where a deflectable tab 347 is used to block inner lumen 302. Here, tab 347 is formed from body 301 and deflected into inner lumen 302 to reduce the possibility of shunting.

Clip 103 is preferably fabricated from a superelastic material such as NITINOL and the like or an elastic material such as stainless steel and the like, so as to provide the desired biased deflections or shape altering characteristics. Any shape memory characteristics of the material (e.g., NITINOL) can also be incorporated into the functional operation of clip 103. For instance, in one exemplary embodiment, body 301 is composed of NITINOL and heat treated in the deployed configuration so as to instill that shape. A typical heat treatment procedure can occur for 1-20 minutes in a temperature range of 500-550° C. based on factors such as the heating device and the clip material, although clip 103 is not limited to heat treatment in only that range of time and temperature. The process steps and conditions for heat treating NITINOL to instill a desired shape is well known to those of ordinary skill in the art. After heat treatment, members 306 and 307 become biased towards the deployed configuration such that members 306 and 307 will remain deformable yet will resist any deflection or movement away from that configuration. Members 306 and 307 can then be deflected into the undeployed configuration so that clip 103 can be loaded into delivery device 104 (e.g., needle 120, sheath 123, etc.). Therefore, upon exposure of clip 103 from within delivery device 104, members 306 and 307 will begin to return to the heat-treated, deployed configuration.

FIG. 5G is a partial cross-sectional view depicting the proximal portion of an exemplary embodiment of clip 103 located within lumen 122 of needle 120. Also located within lumen 122 is pusher member 128, which is depicted by itself in the perspective view of FIG. 5H. In this embodiment, pusher member 128 is a tubular, elongate member having an inner lumen 129, distal end 130 (shown in FIG. 5H) and outward extending tabs 131. Tabs 131 are configured to engage clip 103 and allow clip 103 to be moved distally and proximally within lumen 122. In this embodiment, tabs 131 extend into inner apertures 349 of annular end tips 315. In addition to allowing both distal and proximal movement, this configuration also allows rotational movement and orientation of clip 103 through rotation of pusher member 128.

The embodiment of pusher member 128 depicted in FIG. 5H includes multiple apertures 132 that allow pusher member 128 to more easily bend to accommodate the preferred off-axis delivery method and any tortuous vasculature through which pusher member 128 is routed while within delivery device 104. Tether 316 can be routed through one of these apertures 132 or through an additional aperture (not shown). Tether 316 can also be routed through one or more annular end tips 315, or any other aperture on clip 103, as will be discussed in more detail below. If, removal of clip 103 is desired after partial or complete deployment, pusher member 128 can be proximally retracted to pull tether 316, which in turn will pull clip 103 from septal wall 207 back into the desired portion of delivery device 104. Tether 316 can be fabricated UHMWPE (Ultra High Molecular Weight Polyethylene) or KEVLAR (poly-paraphenylene terephthalamide) or any other material having a relatively high tensile strength.

FIGS. 5I-J are perspective views depicting another exemplary embodiment of pusher member 128. Here, pusher member 128 is configured to exert a spring-like force in directions 416 to maintain tabs 131 in an engaged position within apertures 349 of clip 103 (not shown). Pusher member 128 has two opposing slots 417 that allow the distal end portions 418 of pusher member 128 to deflect outwards in directions 416. FIG. 51 depicts pusher member 128 with portions 418 in an undeployed configuration, while FIG. 5J depicts portions 418 in a deployed configuration. This embodiment of pusher member 128 can be fabricated from any desired superelastic material, such as NITINOL and the like, or elastic material, such as stainless steel and the like. If made of NITINOL, pusher member 128 is preferably heat treated in the deployed configuration so that portions 418 are biased to deflect to that configuration. The force exerted by portions 418 can be customized by varying the length and width of slots 417 as well as the wall thickness of portions 418. This force is preferably sufficient to maintain engagement with clip 103 until after deployment from within needle 120, at which point clip 103 will self-release as RA members 307 enter their outwardly deflected configuration.

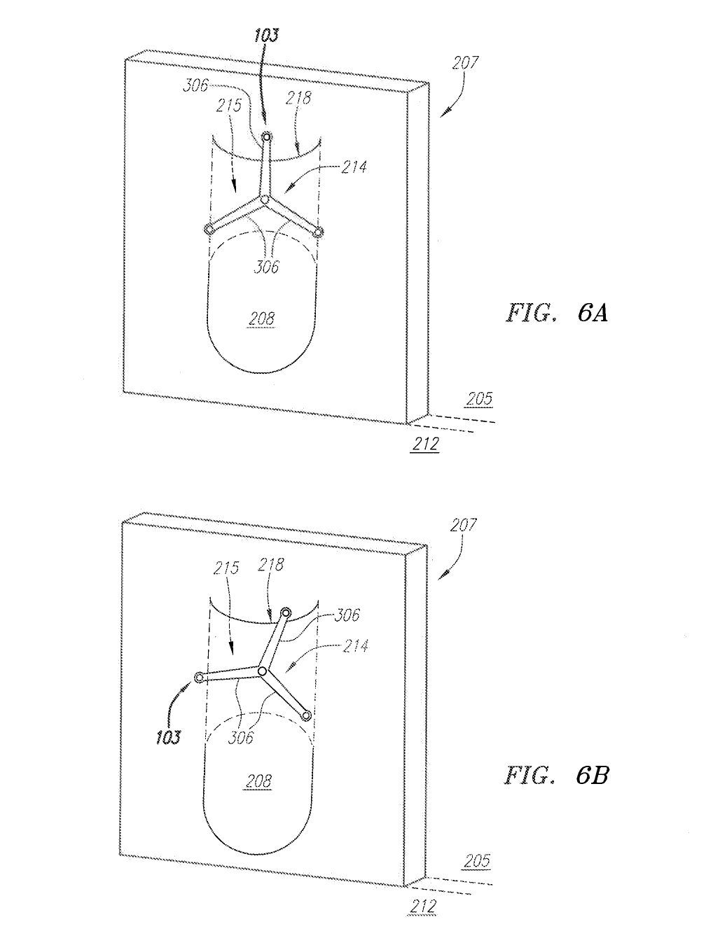

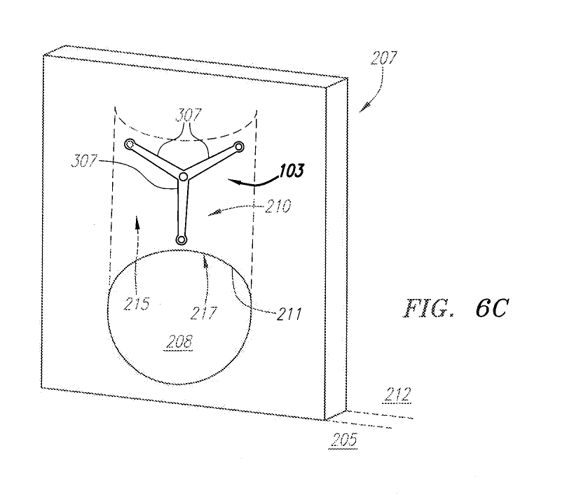

FIGS. 6A-C are perspective views depicting an exemplary embodiment of clip 103 implanted within septal wall 207. FIG. 6A depicts clip 103 within septal wall 207 as viewed from left atrium 212. Here, it can be seen that LA members 306 are configured to extend over a relatively wide surface area, preferably overlapping both the PFO tunnel 215 and a portion of the adjacent non-tunneled septal wall 207, while avoiding placement over fossa ovalis 208. It can also be seen that varying degrees of rotation of this embodiment of clip 103 will result in varying degrees of overlap as shown in FIG. 6B, where the embodiment of clip 103 has a different rotational orientation than in FIG. 6A. This different orientation provides greater overlap on the left side of tunnel 215, but no overlap on the right side of tunnel 215.

FIG. 6C depicts this embodiment of clip 103 within septal wall 207 as viewed from right atrium 205. Due to the relatively shorter RA members 307, the surface area covered by RA members 307 is relatively less than that covered by LA members 306. Because septum secundum 210 is typically a thicker, more rigid tissue flap than septum primum 214, a high degree of surface area coverage over septum secundum 210 is not needed to adequately engage and maintain the desired location on secundum 210. On the other hand, septum primum 214 is typically a thin, floppy, and mobile tissue flap, so a relatively high degree of surface area coverage is preferable to achieve proper closure. FIG. 6C depicts clip 103 oriented such that one RA member 307 extends over limbus 211, thereby providing added closure force to PFO entrance 217.

The optimal orientation of clip 103 is dependent on numerous factors, some of which can include the actual configuration and implementation of clip 103, such as the number and shape of LA and RA arms 306 and 307, the placement of opening 206 and the nature of the PFO region 209 itself, to name a few. In general, clip 103 can be configured to avoid certain types of contact, such as intrusion, into potentially sensitive areas of the anatomy, such as fossa ovalis 208 and septum primum 214, or clip 103 can be configured to have substantial contact with potentially more stabile areas of the anatomy, such as septum secundum 210 and limbus 211.

As mentioned above, clip 103 is preferably fabricated from an elastic, shape memory material such as NITINOL and the like. Clip 103 can be fabricated in any manner desired. In one exemplary embodiment, clip 103 is formed from a NITINOL tube, which is laser cut into the desired clip shape, such as that of the undeployed configuration depicted in FIG. 5B. In another embodiment, a pattern of clip 103 is formed from a NITINOL (or other shape memory material) sheet and then shaped into clip 103. In this case, the sheet can be molded or formed directly into the clip pattern, or the clip pattern can be formed by separating it from the sheet in any manner desired including, but not limited to, laser cutting, etching, sawing, stamping and the like. The separated pattern can then be shaped or rolled into a tubular configuration. Clip 103 can be post-processed after being separated from the sheet. This post-processing can include smoothing any sharp or rough edges located on clip 103 with processes such as electro-polishing and the like.

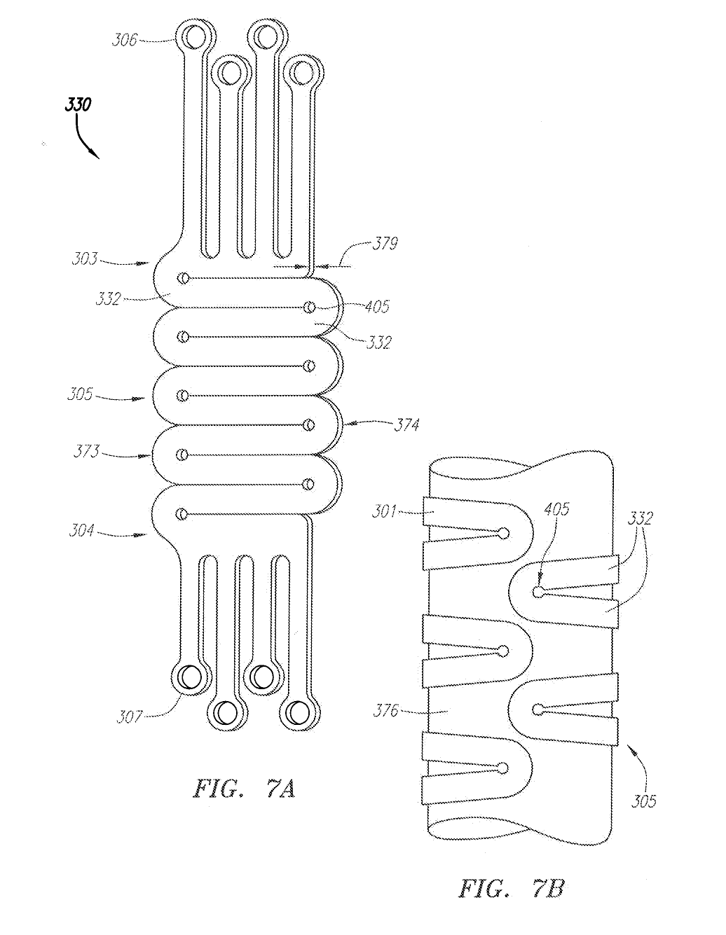

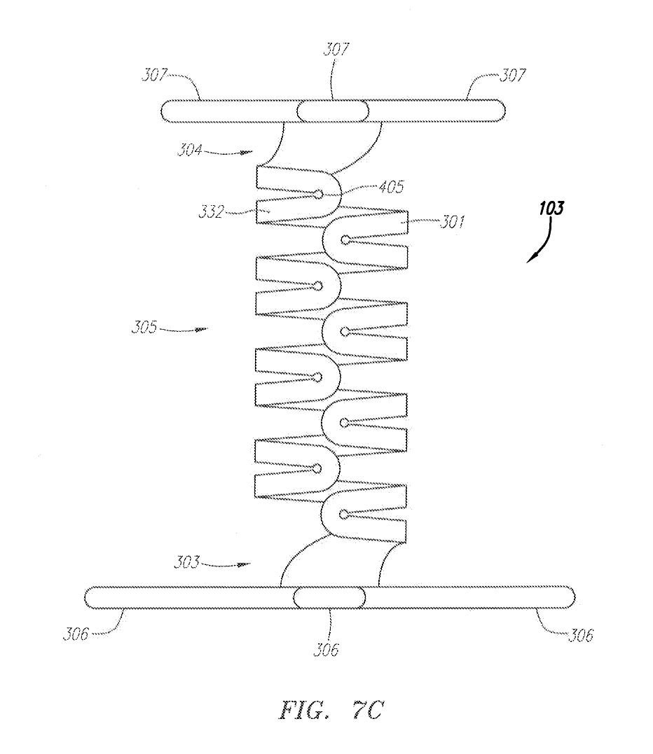

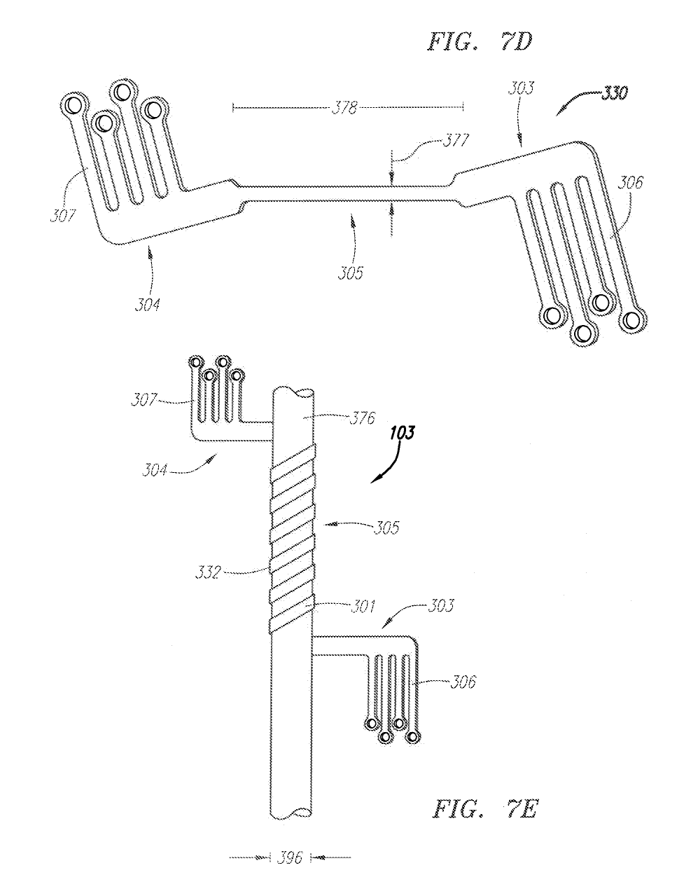

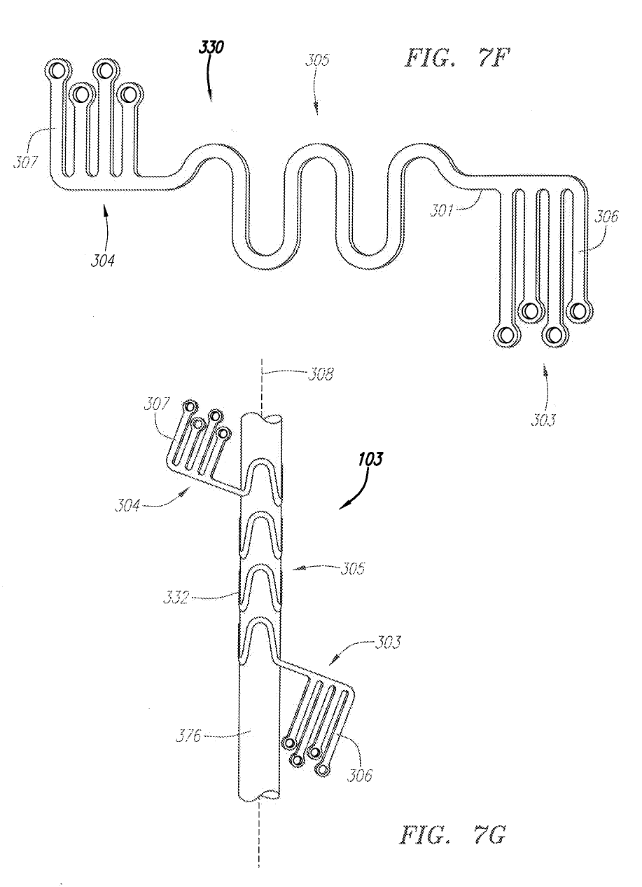

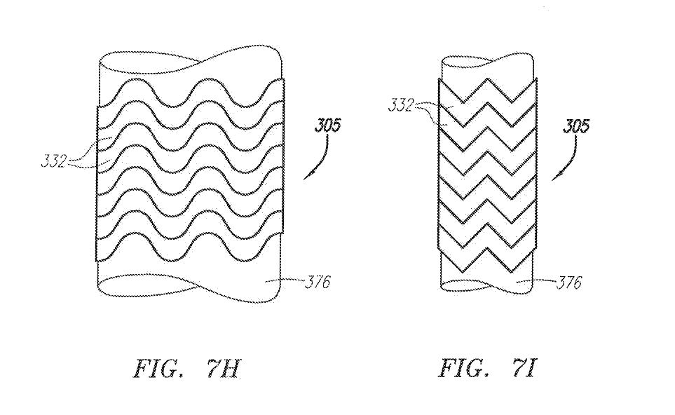

FIG. 7A depicts a NITINOL sheet 330 after being laser cut to form the desired shape for forming clip 103. Here, sheet 330 has a thickness 379 in the range of 0.005-0.010 inches, although sheet 330 is not limited to such and any thickness 379 can be used. Photo-etching, chemical etching and other techniques can be used to vary the thickness 379 of sheet 330 in predetermined locations. For instance, sheet 330 can be relatively thinner in central portion 305 and relatively thicker in end portions 303-304.

Sheet 330 can be rolled up so that sides 373 and 374 are in proximity with each other to create clip 103. In this embodiment, coiled segments 332 in central portion 305 are wrapped back and forth between sides 373 and 374 to create continuous “S” shapes. Each segment 332 has an aperture 405 to allow flexing and stress relief. To hold clip 103 in the tubular configuration, sides 373 and 374 can be fixably coupled together in any manner desired, such as with adhesive, welding, soldering, interlocking tabs and the like. Alternatively, sheet 330 can be heat treated to maintain the tubular configuration without the need to fixably couple sides 373 and 374 together. In FIG. 7B, central portion 305 of clip 103 is shown wrapped around a mandrel 376 for heat treatment. Heat treatment to place clip 103 in the tubular configuration and to instill the deflection to members 306 and 307 can occur separately or substantially simultaneously. FIG. 7C is a frontal view depicting this embodiment of clip 103 in an expanded, deployed configuration.

Clip 103 can also be fabricated from sheet 330 using helical or other configurations of coiled central portion 305. FIG. 7D is a perspective view depicting one exemplary embodiment of clip 103 formed from sheet 330 and prior to shaping into the coiled clip configuration. Here, central portion 305 is an elongate strip having a width 377 and length 378. In this embodiment, width 377 is relatively greater in the areas adjacent end portions 303 and 304 than in the central portion 305 in order to provide the desired level of compliance (i.e., expandability/compressibility) to central portion 305 and the desired support to end portions 303 and 304. FIG. 7E depicts this embodiment of clip 103 partially wrapped around mandrel 376 during the fabrication process. In this embodiment, mandrel 376 is circular and has a diameter 396 that determines diameter 317 of clip 103. Diameter (or width) 396 of mandrel 376 can be varied to provide a variable diameter (or width) 317 to clip 103. For instance, the central portion of mandrel 376 corresponding can be made relatively wider or thinner than the end portions of mandrel 376.

FIG. 7F depicts another exemplary embodiment of clip 103 prior to winding around mandrel 376. Here, central portion 305 is curved in a serpentine-type shape. FIG. 7G depicts this embodiment of clip 103 partially wrapped around mandrel 376. This configuration of central portion 305 can allow additional flexibility in a lateral direction perpendicular to main axis 308. It should be noted that central portion 305 can be shaped in any manner desired. FIGS. 7H-I are frontal views depicting additional exemplary embodiments of central portion 305 wrapped around mandrel 376. In FIG. 7H, central portion 305 is a curved, serpentine-like shape wound relatively tighter than the embodiment depicted in FIG. 7G, while central portion 305 depicted in FIG. 71 has a “zig-zag” type shape. Central portion 305 of clip 103 can also be configured with any stent-type shape desired, such as shapes used to fabricate medical stents used in interventional cardiology procedures.

Clip 103 can also be configured such that body 301 is split into multiple body elements in one or more of portions 303-305. FIG. 7J is a frontal view depicting an exemplary embodiment of clip 103 where body 301 splits into multiple body elements 419 in the center of portion 305. Each element is connected only at the center of clip 103 and each element also has multiple LA members 306 or RA members 307 coupled thereto. FIG. 7K is a frontal view depicting another exemplary embodiment of clip 103 where body 301 is continuous within end portions 303 and 304 but splits into multiple elements 419 along central portion 305. Both of these embodiments provide added flexibility to clip 103.

NITINOL can be an anisotropic material, meaning that it has properties (e.g., Young's modulus, percent elongation at break, tensile strength, etc.) that are not identical in all directions but are a function of the orientation of the material. The anisotropic properties of NITINOL are preferably taken into account when fabricating clip 103. For instance, when forming LA members 306 and RA members 307, the orientation of the NITINOL material (e.g., sheet, tube, rod, etc.) can be adjusted to maximize the flexibility, deflectability and the like.

Any portion of clip 103 can be coated with any material as desired. Some exemplary coatings that can be used include coatings that are biodegradable, drug coatings (e.g., drugs can be released from hydrogels or polymer carriers where the polymer itself is a biodegradable material (e.g., poly(caprolactone), poly(D,L-lactic acid), polyorthoester, polyglycolides, polyanhydrides, erodable hydrogels and the like) or elastomers (e.g., polyurethane (PU), polydimethylsiloxane (PDMS) and the like), coatings that increase or decrease lubricity (e.g., hydrogels, polyurethane and the like), bioactive coatings (e.g., anti-platelet coatings, anti-microbial coatings and the like), coatings that inhibit thrombus formation or the occurrence an embolic events (e.g., heparin, pyrolytic carbon, phosphorylcholine and the like), and coatings that speed the healing response.

These coatings can be applied over the entire clip 103 or any portion thereof. Also, different portions of clip 103 can be coated with different coatings. For instance, because end portion 303 and LA members 306 lie within left atrium 212 in contact with the oxygenated arterial blood, it may be desirable to coat that region of clip 103 with a material designed to inhibit thrombus formation. On the other hand, end portion 304 and RA members 307 lie within right atrium 205 in contact with the oxygen-depleted venous blood, and it may therefore be desirable to coat that region of clip 103 with a material designed to accelerate or promote the healing response.

Clip 103 can also be coated in layers. For instance, in one exemplary embodiment clip 103 has two coatings applied: a first, underlying coating and a second coating situated over the first coating and exposed to the surrounding environment. The second, exposed coating can be a short term coating designed to dissolve over a desired time period. The second coating eventually dissolves enough to expose the underlying first coating, which can itself be configured to dissolve or can be a long term, permanent coating. Any number of coatings having any desired absorption rate or drug elution rate can be used.

Any portion of clip 103 can be made easier to view by an internal or external imaging device. For instance, in one embodiment radio-opaque markings are added to members 306 and 307 to make clip 103 viewable via fluoroscopy, while in another embodiment an echolucent coating is added to make clip 103 viewable with ultrasound devices. Clip 103 can be configured for use with any internal or external imaging device such as magnetic-resonance imaging (MRI) devices, computerized axial tomography (CAT) scan devices, X-ray devices, fluoroscopic devices, ultrasound devices and the like.

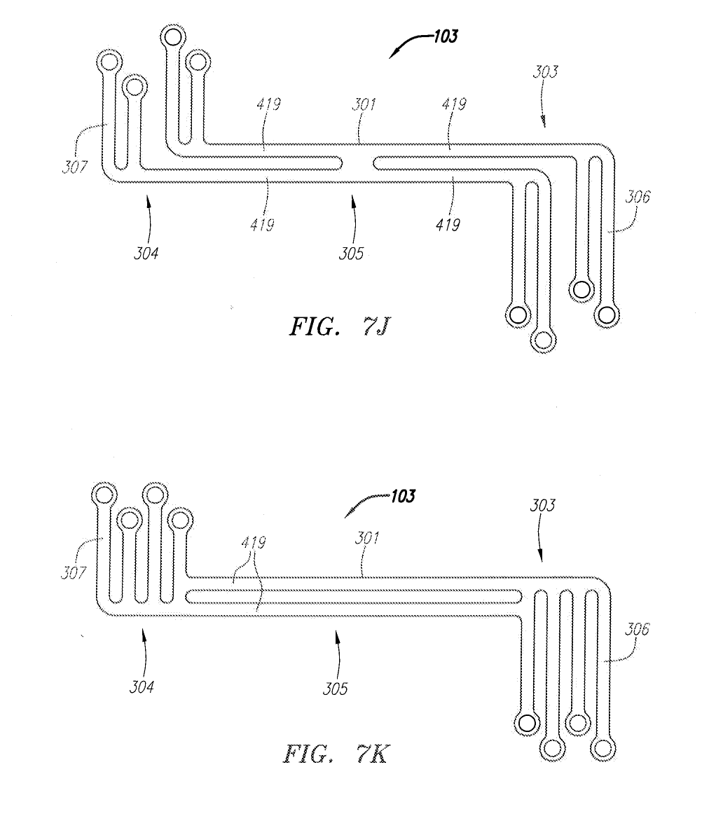

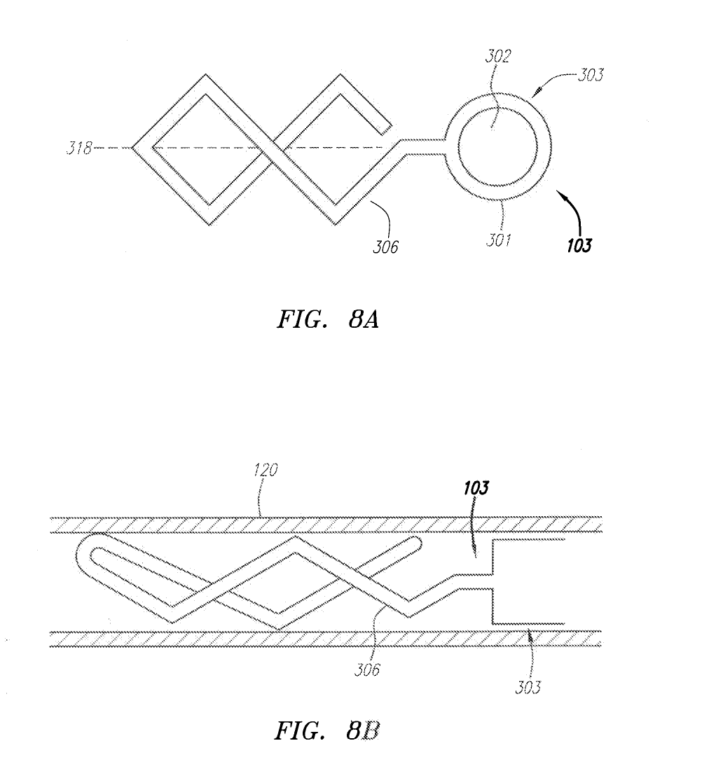

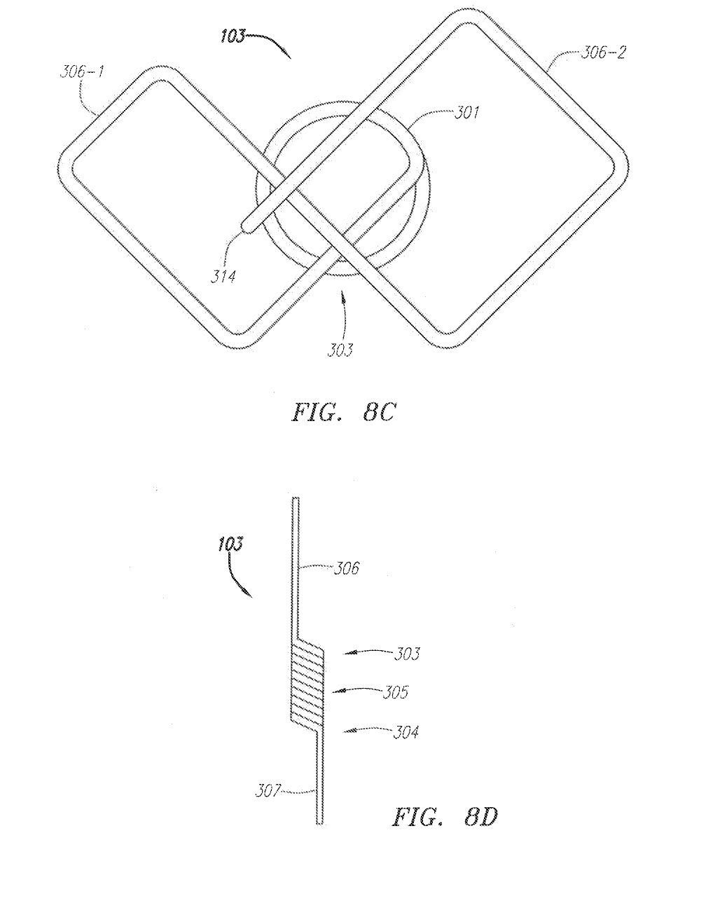

As mentioned above, clip 103 can be configured in numerous different variations. The following discussion and FIGS. 8A-17B further describes the many different variations in which LA and RA members 306 and 307 can be configured. For instance, in the above embodiments, LA and RA members 306 and 307 are depicted as being relatively straight, flap-like or petal-like members. However, LA and RA members 306 and 307 can have any shape or structure configured to deflect and abut the desired tissue surface. FIG. 8A is an end-on view depicting an exemplary embodiment of clip 103 with one LA member 306 having a curved or bent shape in the deployed configuration. It should be noted that this same configuration could also be applied to RA member 307. Here, LA member 306 deflects into a predetermined configuration, resembling a “bow-tie” (shown with longitudinal axis 318), which can cover a relatively greater surface area than one relatively straight, flap-like member 306. FIG. 8B depicts this embodiment of member 306 in the undeployed configuration within needle 120.

FIG. 8C is an end-on view of end portion 303, depicting an additional exemplary embodiment of clip 103 in the deployed configuration where LA members 306 are configured in a “bow-tie” fashion. Here, LA members 306 are formed from one continuous elongate section that extends outwards to form a first LA member 306-1 and then crosses over end portion 303 and forms a second LA member 306-2. In this embodiment, end tip 314 lies on top of the remainder of the continuous elongate section. Clip 103 can also be configured so that end tip 314 resides underneath the continuous elongate section after deployment to provide additional strength to LA members 306-1 and 306-2. FIG. 8D is a frontal view of an embodiment of clip 103 in the undeployed configuration where LA members 306 and RA members 307 are formed into a “bow-tie” fashion.

In the description herein, multiple instances of the same or similar elements that are distinguished from each other are done so using the notation YYY-X, where Y is the reference numeral of the element and X is used to identify a specific one of the multiple instances of the element.

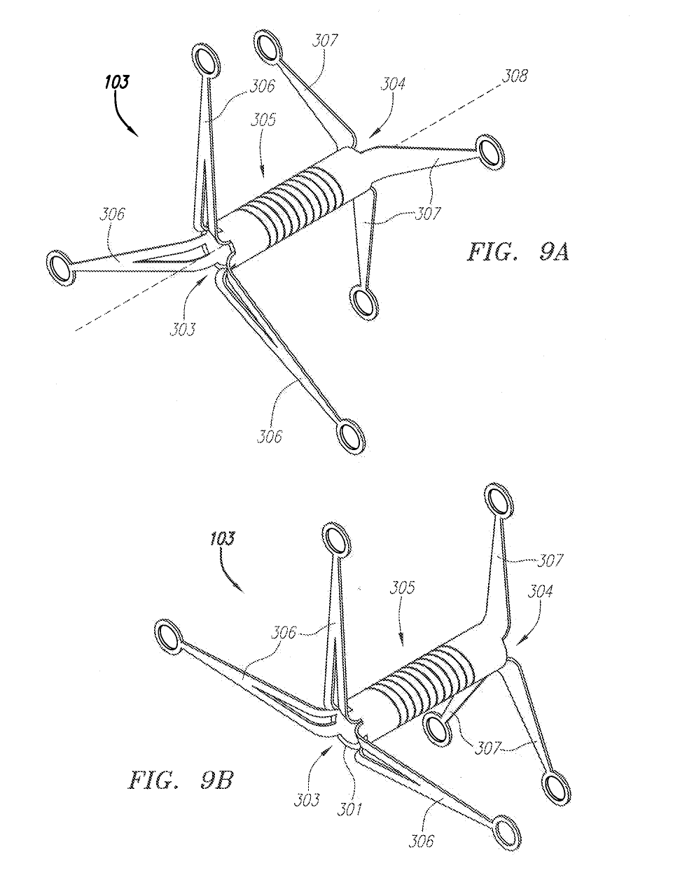

FIG. 9A is a perspective view depicting an exemplary embodiment of clip 103 having LA members 306 having different orientations than RA members 307. Here, each RA member 307 is offset by approximately sixty degrees about main axis 308 with respect to LA members 306. Variation of the orientation of LA members 306 and RA members 307 can allow for greater closing force and can accommodate for differing tissue characteristics. For instance, it may be desirable to avoid placement of an LA member 306 over fossa ovalis 208, in order to mitigate the risk of inadvertently puncturing the thin fossa tissue. Accordingly, the orientation that is optimal for LA members 306 may not be optimal for RA members 307, in which case it can be desirable to offset RA members 307 to the desired orientation without affecting LA members 306. In addition to offsetting LA members 306 from RA members 307, LA and RA members 306 and 307 can be placed asymmetrically on end portions 303 and 304, respectively, as depicted in FIG. 9B. Asymmetric placement allows additional freedom in orienting members 306 and 307.

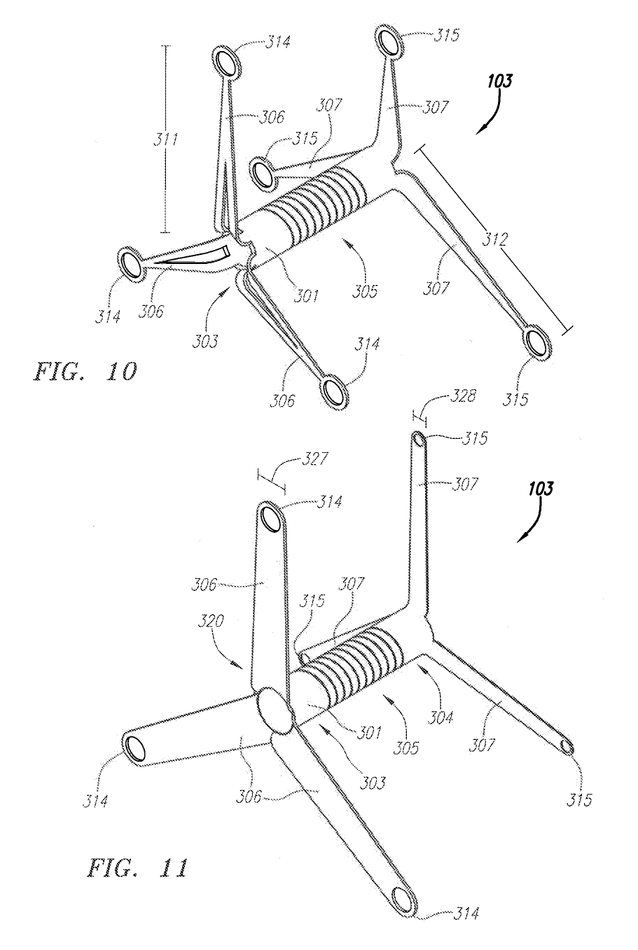

FIG. 10 is a perspective view depicting another exemplary embodiment of clip 103 where LA members 306 and RA members 307 have varying lengths 311 and 312, respectively. Variation of these lengths 311 and 312 can again allow for optimal placement of LA and RA members 306 and 307. For instance, LA members 306 in proximity with fossa ovalis 208 can be made relatively shorter to avoid contact with and the inadvertent puncturing of fossa ovalis 208. In addition, lengths 311 and 312 can be adjusted to allow room for larger end tips 314 and 315 when in the undeployed configuration. Length variation can also provide control to the order in which RA members 307 are deployed. For instance, relatively shorter members 307 will be exposed from within needle 120 and become free to deploy before relatively longer members 307.

In addition to varying lengths 311 and 312, the widths 327 and 328 of each LA and RA member 306 and 307 can be along lengths 311 and 312, respectively, as desired. FIG. 11 is a perspective view of another exemplary embodiment of clip 103. Here, the width 327 of each LA member 306 is relatively greater than the width 328 of each RA member 307, for instance, in order to provide added strength to LA members 306. Widths 327 and 328 along each LA and RA member 306 and 307 can also be variable. Here, each LA member 306 has a variable width 327 that decreases from base 320 to end tip 314.

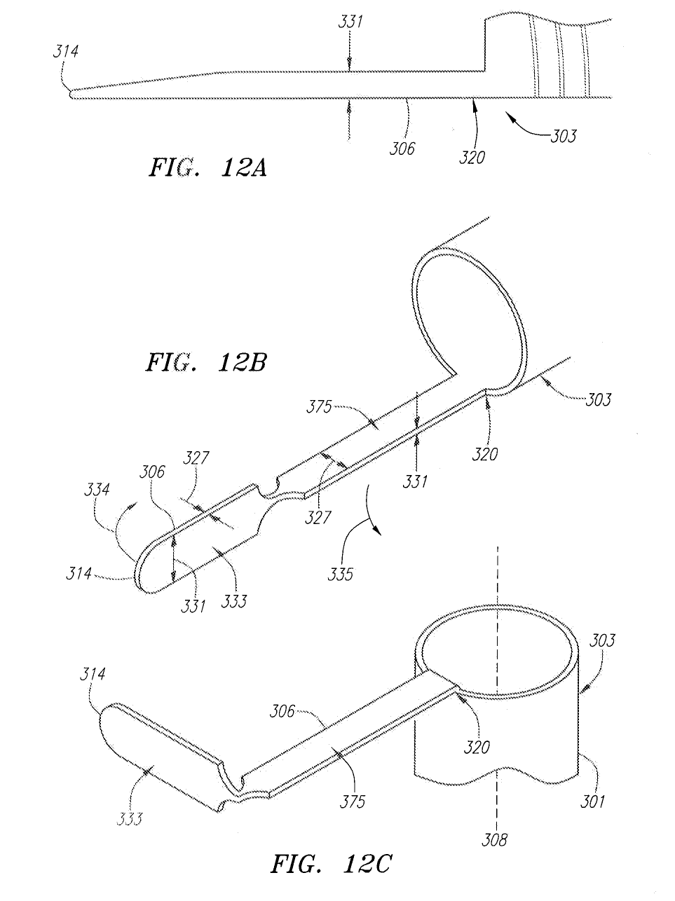

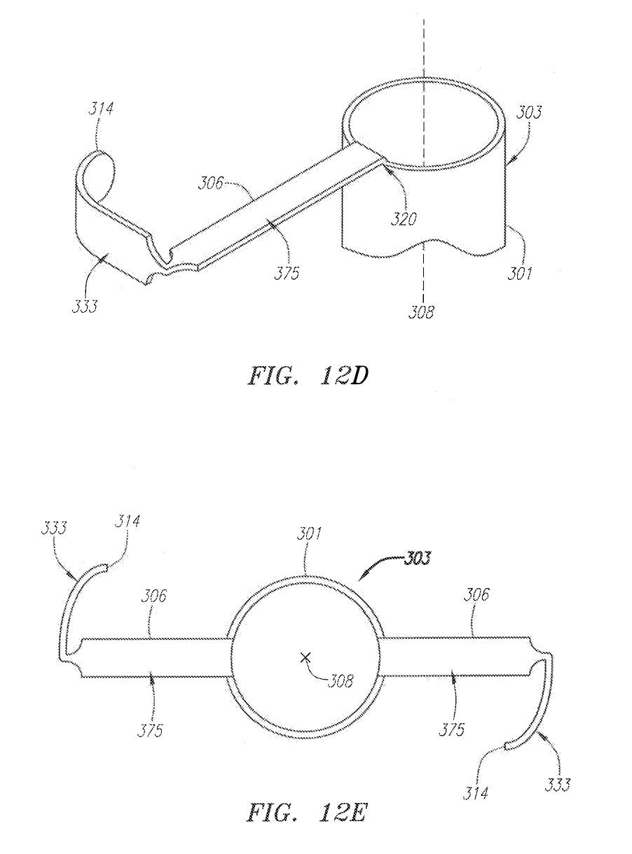

Also, the thickness of each LA and RA member 306 and 307 can be varied along lengths 311 and 312, respectively, as desired. Thickness variations can effect the strength of members 306 and 307 as well as the position in which the member 306 or 307 will be more or less likely to bend. FIG. 12A is a perspective view of one exemplary LA member 306 having a tapered thickness 331 that is relatively constant adjacent base portion 320 and then becomes progressively thinner approaching end tip 314. This thinner region can make end tip 314 more easily deformable so as to be atraumatic to any adjacent tissue, whereas the thicker base portion 320 is relatively stronger and more rigid to maintain an adequate amount of closure force onto PFO tunnel 215.

FIG. 12B is a perspective view of another exemplary LA member 306 having regions of varying thickness 331 and width 327. First region 375, which is adjacent to base portion 320, has a relatively large width 327 and a relatively small thickness 331. Second region 333, which is between first region 375 and end tip 314, has a relatively small width 327 and a relatively large thickness 331. These combinations of width 327 and thickness 331 allow member 306 to more easily deflect in directions 334 and 335. FIG. 12C is a perspective view depicting end portion 303 with this embodiment of LA member 306 in the deflected, deployed configuration. This configuration allows LA member 306 to be less traumatic to septal wall 207 and also allows LA member 306 to contact more surface area of septal wall 207 without extending further from main axis 308.

FIG. 12D is a perspective view depicting another exemplary embodiment of clip 103 similar to that depicted in FIG. 12C. Here, region 333 is curved inwards towards main axis 308 to make LA member 306 more atraumatic to any surrounding tissue. FIG. 12E is an end-on view of this embodiment of clip 103 showing curved regions 333 on two opposing LA members 306.

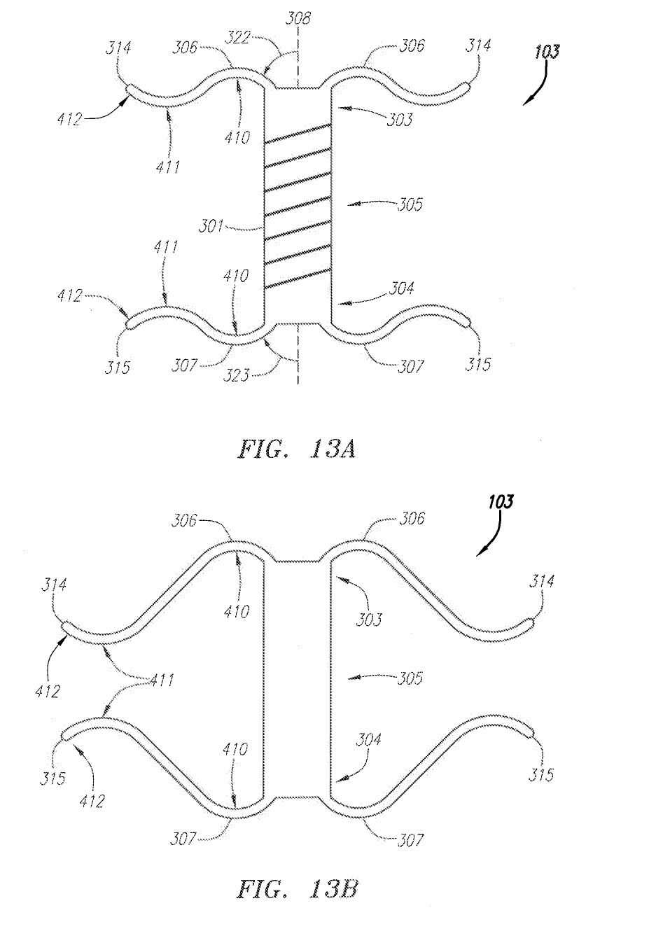

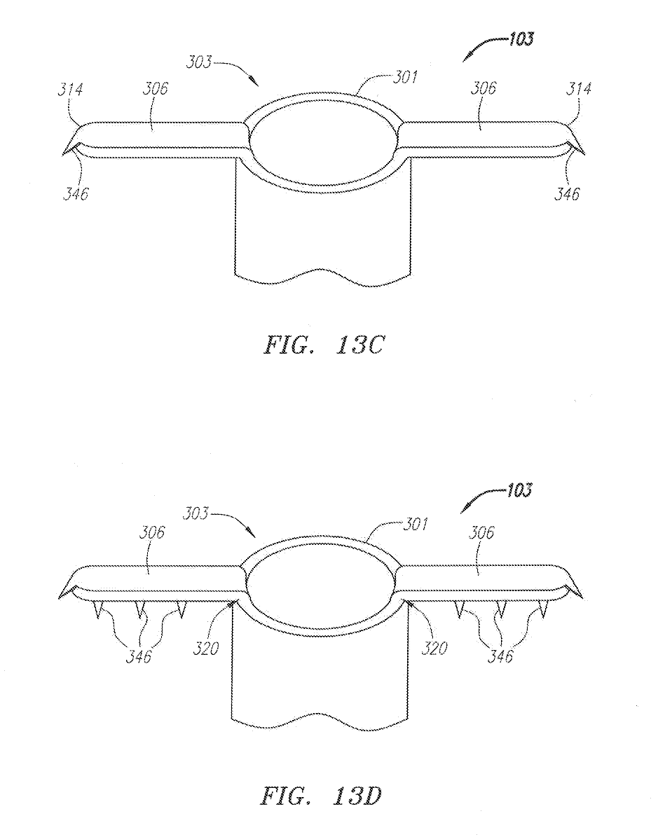

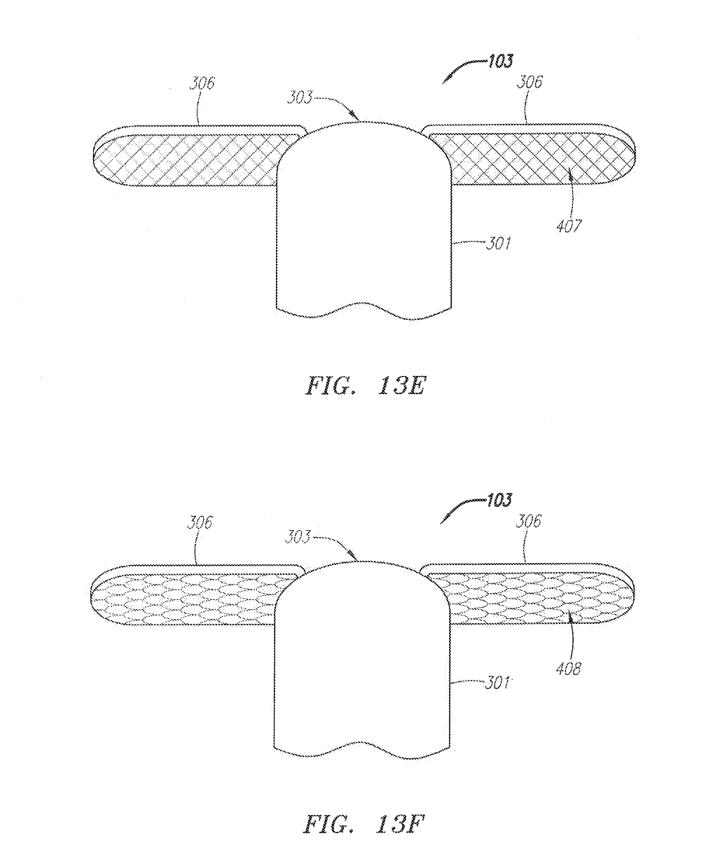

FIG. 13A is a frontal view of another exemplary embodiment of clip 103 in the deployed configuration, where LA and RA members 306 and 307 have a varying, non-flat surface. Here, the LA and RA members 306 and 307 have a curved, wave-like surface to conform to septal wall 207. LA and RA members 306 and 307 each include an inner curved portion 410, an intermediate curved portion 411, and an outer curved portion 412, referenced from the relative position in the deployed configuration with respect to main axis 308. With respect to LA members 306, deflection angle 322 of inner curved portion 410 is preferably less than ninety degrees. This can serve one of at least two functions. First, the smaller deflection angle 322 may be preferred in order to adhere to the minimum bend radius of the constituent material of LA members 306. Second, this smaller deflection angle 322 can accommodate the septal tissue surrounding end portion 303, which may be pushed outward and/or swollen due to the creation of opening 206 and the implantation of clip 103 therein.

In the deployed configuration, intermediate curved portion 411 extends towards the opposite end of clip 103 and can be used to press against septal wall 207 and apply a closure force thereto. This closure force can be in addition to the closure force applied by central portion 305. Outer curved portion 412 extends back away from the opposite side of clip 103 so that end tip 314 does not extend into septal wall 207 and increase the risk of septal wall 207 perforation.

FIG. 13B is a frontal view depicting another embodiment where central portion 305 is not compressive and the entire closure force is generated by LA and RA members 306 and 307, which extend towards each other such that they are in relatively close proximity. Generally, more closure force can be generated and a wider range of septal wall thicknesses can be accommodated the further that intermediate portion 411 extends towards the opposite end of clip 103. Although not shown, end tips 314 and 315 can be magnetized or configured to carry magnets that create an attractive force between end tips 314 and 315 and generate additional closure force.

It should be noted that LA and RA members 306 and 307 can have any type of surface configured for any desired purpose including, but not limited to, increasing engagement with septal wall 207, conforming to septal wall surfaces and the like. For instance, in another exemplary embodiment, an RA member 307 can be configured to conform to and wrap over limbus 211. Generally, the ability to conform to septal wall 207 is desirable because it minimizes the amount in which clip 103 sits exposed in the blood flow path, thereby minimizing the risk of clotting and thrombus embolization.

LA and RA member end tips 314 and 315 can also be configured to achieve added functionality as desired. Although preferably atraumatic, end tips 314 and 315 can be configured to increase the surface friction between clip 103 and the surrounding tissue. For instance, FIG. 13C is a perspective view of another exemplary embodiment of end portion 303 of clip 103. Here, end tips 314 each have a tine, or protruding spike 346, configured to engage and grasp septum primum surface 213. Tine 346 is preferably small enough to avoid significant tissue injury and is preferably used on any LA end tips 314 not in proximity with fossa ovalis 208.

Tine 346 can be located in any position on body 301 where it is desirable to increase the surface friction with adjacent tissue. FIG. 13D is a perspective view of another exemplary embodiment of end portion 303 of clip 103 where LA member 306 has multiple tines 346 located between base portion 320 and end tip 314. Also, the surface of LA members 306 can be textured to increase friction with the underlying septal tissue. FIGS. 13E-F are perspective views of additional exemplary embodiments of end portion 303 of clip 103 where LA member 306 has a cross-hatched surface texture and a fish scale-type surface texture, respectively. In light of this disclosure, one of ordinary skill in the art will readily recognize the numerous various surface configurations and textures that can be used with clip 103. It should be noted that the these surface configurations and textures described with respect to FIGS. 13C-F can be equally applied to RA members 307.

FIG. 14A is a perspective view of another exemplary embodiment of clip 103 in the undeployed configuration. Here, LA and RA members 306 and 307 are integrated within portions 303 and 304, respectively. LA and RA members 306 and 307 are configured to deflect in directions 329 and 336, respectively, away from central portion 305. FIG. 14B is a frontal view depicting this embodiment in the deployed configuration. Slots 337 and 338 are visible in portions 303 and 304, respectively. Slots 337 and 338 are configured to receive LA and RA members 306 and 307 when in the undeployed configuration. Preferably, LA and RA members 306 and 307 are cut directly from portions 303 and 304 during fabrication.

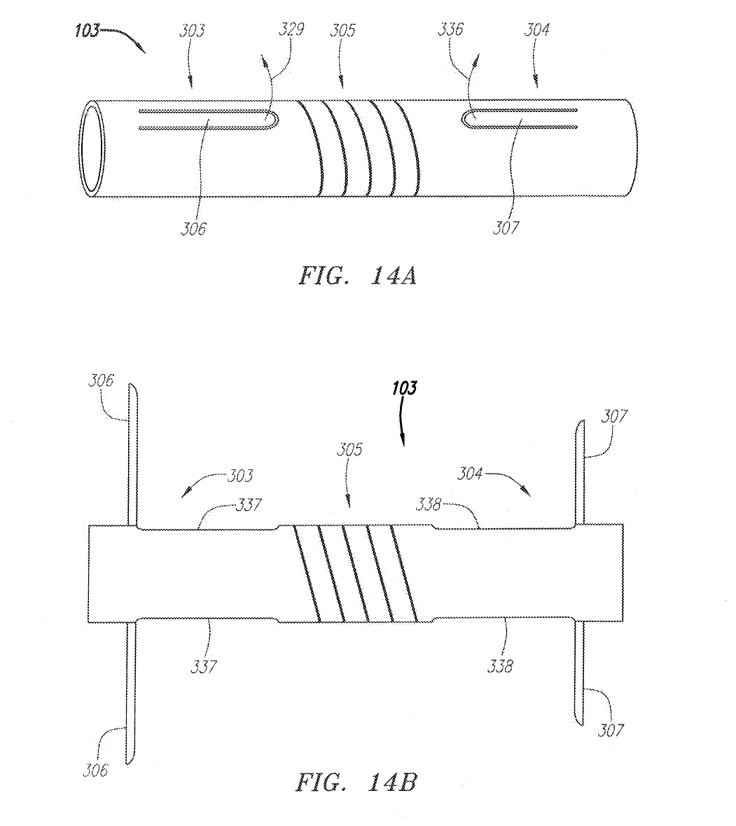

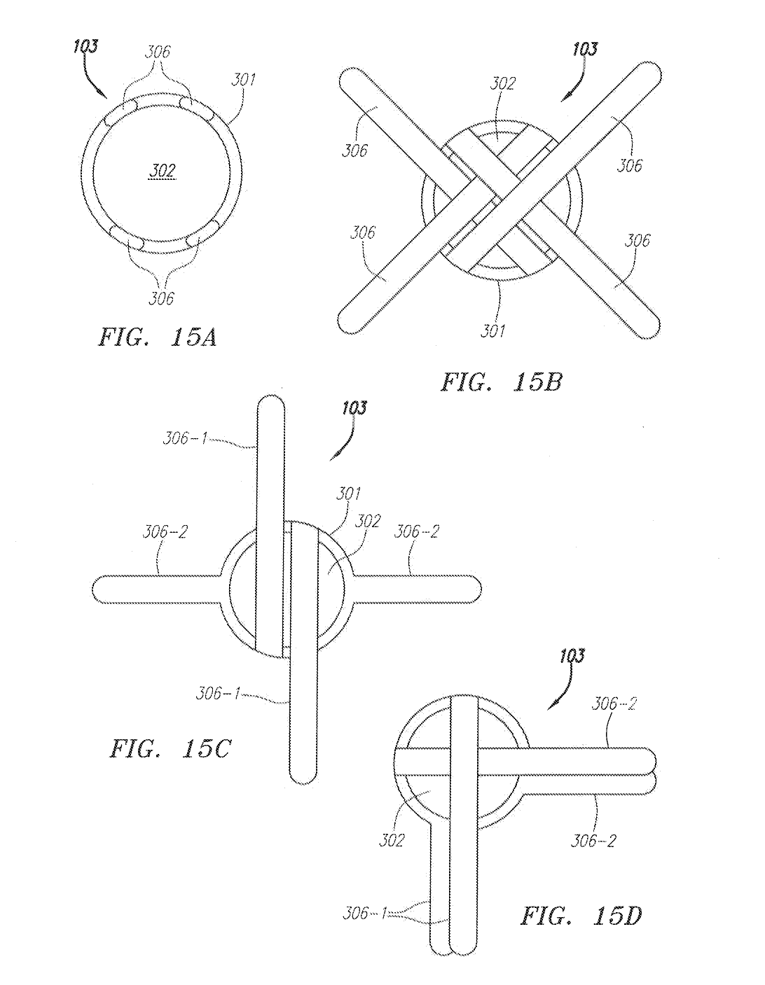

FIGS. 15A-B are end-on views depicting another exemplary embodiment of clip 103 in the undeployed and deployed configurations, respectively. In this embodiment, clip 103 includes four LA members 306, each of which are configured to deflect across end portion 303. When in the deployed configuration, LA members 306 act to reinforce each other to provide added strength and resistance to deflection. In this configuration, LA members 306 also block inner lumen 302 and reduce the likelihood of blood shunting through inner lumen 302. The pressure of the blood within left atrium 212 can also provide additional force to maintain LA members 306 in the deployed configuration. Because each member 306 and 307 deflects inwardly, this configuration also allows delivery of clip 103 without the need to restrain outward deflection of members 306 and 307. For instance, clip 103 could be carried on the outer surface of needle 120, in a manner similar to that depicted in FIG. 4D, except without the use of outer sheath 123.

Because each LA member 306 overlaps end portion 303 and interlocks with other LA members, some care is preferably taken to deploy LA members 306 in a predetermined order. This prevents LA members 306 from “jamming together” in a random fashion. In one embodiment, each LA member 306 has a different length. As clip 103 is deployed from within the needle 120 or other elongate device, the shortest LA member 306 will be exposed first and therefore will deploy first. The shortest of the remaining undeployed members 306 will then deploy next and so on until all members 306 are deployed. In an embodiment where RA members 307 are similarly configured to deploy over inner lumen 302 and end portion 304, the slanted distal end 121 of needle 120 can be used to control deployment of members 307. As needle 120 is retracted proximally, the RA member(s) 307 located adjacent the most proximal portion of needle distal end 121 will deploy first while the RA member(s) 307 located adjacent the most distal portion of needle distal end 121 will deploy later.

FIGS. 15C-D are end-on views depicting additional exemplary embodiments of clip 103 in the deployed configuration having LA members 306 that both do and do not deflect over end portion 303. In FIG. 15C, clip 103 includes a symmetrical arrangement of four LA members 306 where two opposing members 306-1 deflect inwards over lumen 302 and two opposing members 306-2 deflect outwards away from lumen 302. In FIG. 15D, four LA members 306 are arranged in an asymmetric configuration, where a pair of members 306-1 on opposite sides of portion 303 deflect over similar positions, one overlapping end portion 303 and the other not overlapping end portion 303. There is also a similarly configured second pair 306-2. It should be noted that the configurations described with respect to FIGS. 15A-D can also be applied to RA members 307.

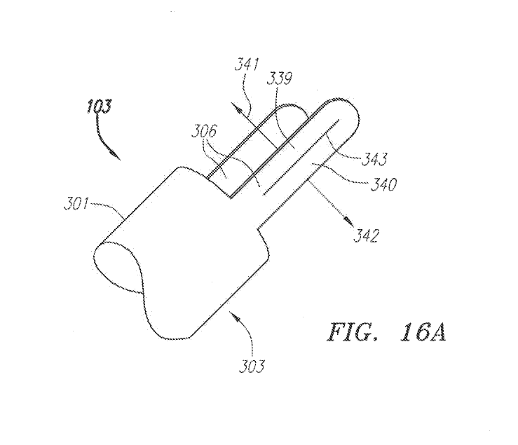

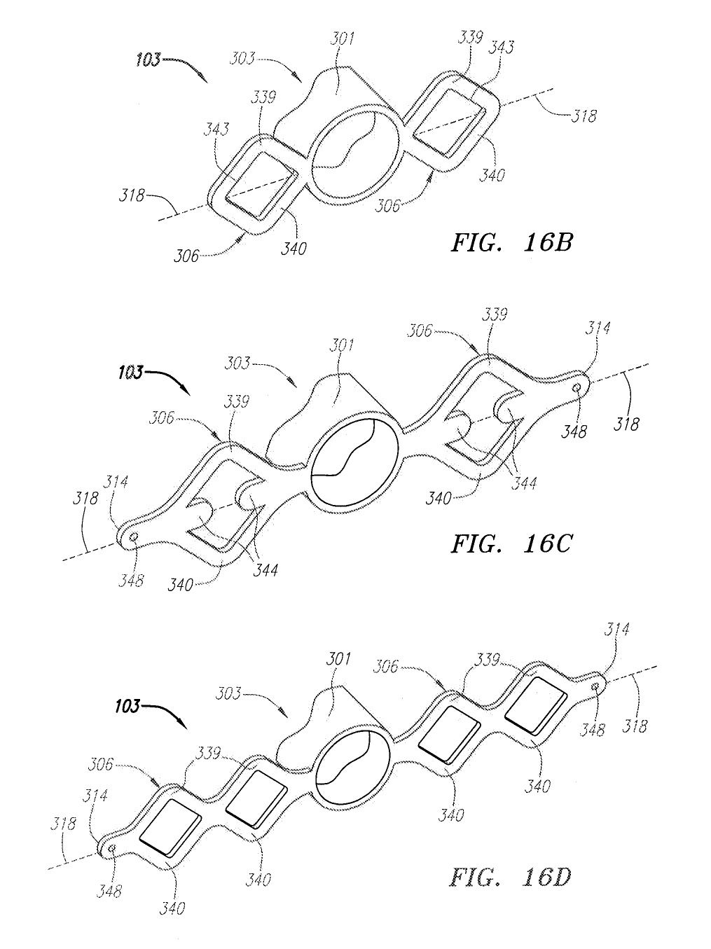

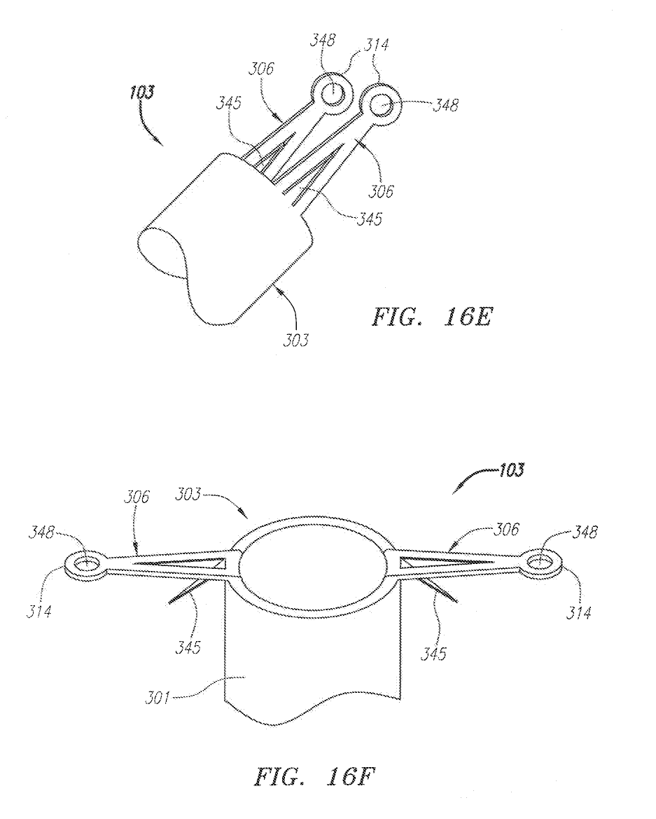

FIGS. 16A-B are perspective views depicting another exemplary embodiment of end portion 303 of clip 103. Here, LA members 306 are configured to expand upon deployment. LA members 306 have deflectable sub-members 339 and 340 that are configured to deflect and allow LA members 306 to cover an expanded surface area region once deployed. FIG. 16A depicts end portion 303 of clip 103 in the undeployed configuration. Sub-members 339 and 340 are biased to deflect away from each other in directions 341 and 342, respectively, once exposed from within needle 120. FIG. 16B depicts end portion 303 of clip 103 in the deployed configuration with sub-members 339 and 340 in their expanded states. In one exemplary embodiment, clip 103 can be fabricated by cutting a slot 343 into each LA member 306. Clip 103 can then be heat-treated in the deployed and expanded configuration such that members 339 and 340 are biased to enter the deployed and expanded configuration from the undeployed configuration after deployment.

FIGS. 16C-D are perspective views depicting additional exemplary embodiments of end portion 303 of clip 103 in the deployed configuration and having expandable LA members 306 with end tip apertures 348. In FIG. 16C, LA members 306 each include a third and fourth opposing sub-members 344 placed between sub-members 339 and 340 and configured to provide additional support against septal wall 207 within the inner open region between sub-members 339 and 340. In FIG. 16D, LA members 306 each include two adjacent pairs of deflectable sub-members 339 and 340. LA members 306 are also shown with longitudinal axes 318 in each of FIGS. 16B-D.

FIGS. 16E-F are perspective views depicting an additional exemplary embodiment of end portion 303 of clip 103. Here, each LA member 306 has a centrally located deflectable sub-member 345. The presence of the centrally located sub-member 345 increases the flexibility of LA member 306. Sub-member 345 can also be biased to deflect if desired. FIG. 16E depicts clip 103 in the undeployed configuration, while FIG. 16F depicts clip 103 in the deployed configuration, with sub-member 345 deflected downwards towards central portion 305 (not shown). This configuration allows LA member 306 to more adequately engage septum primum surface 213.

As one of ordinary skill in the art will readily recognize, LA members 306 can be configured to expand in numerous varying combinations, not just those depicted in FIGS. 16A-F. Also, it should be noted that the configurations of LA members 306 described with respect to FIGS. 16A-F can be equally applied to RA members 307.

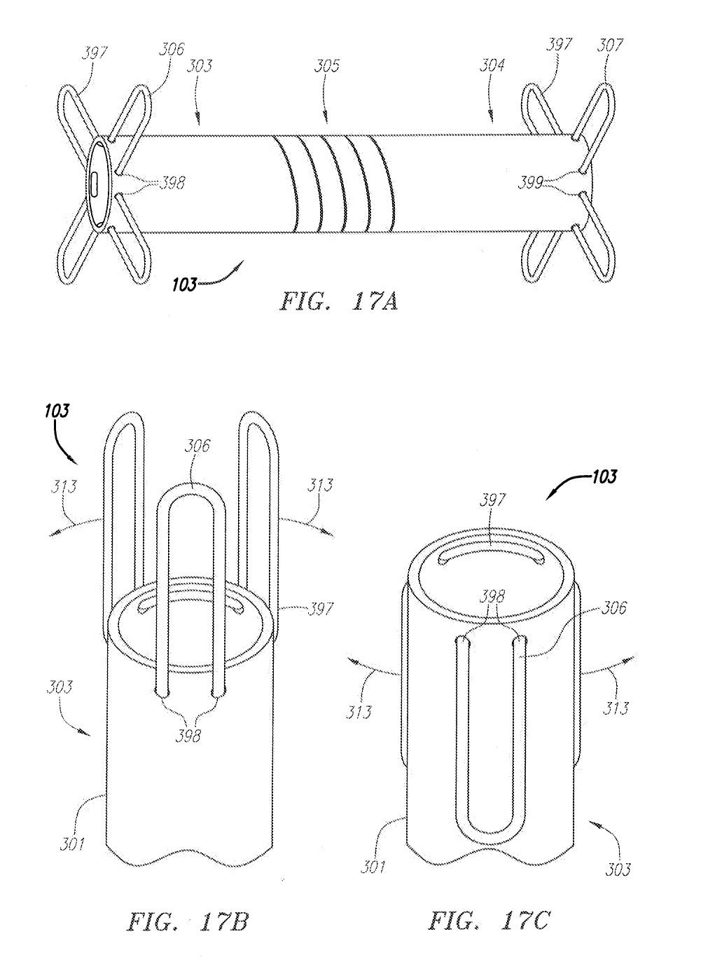

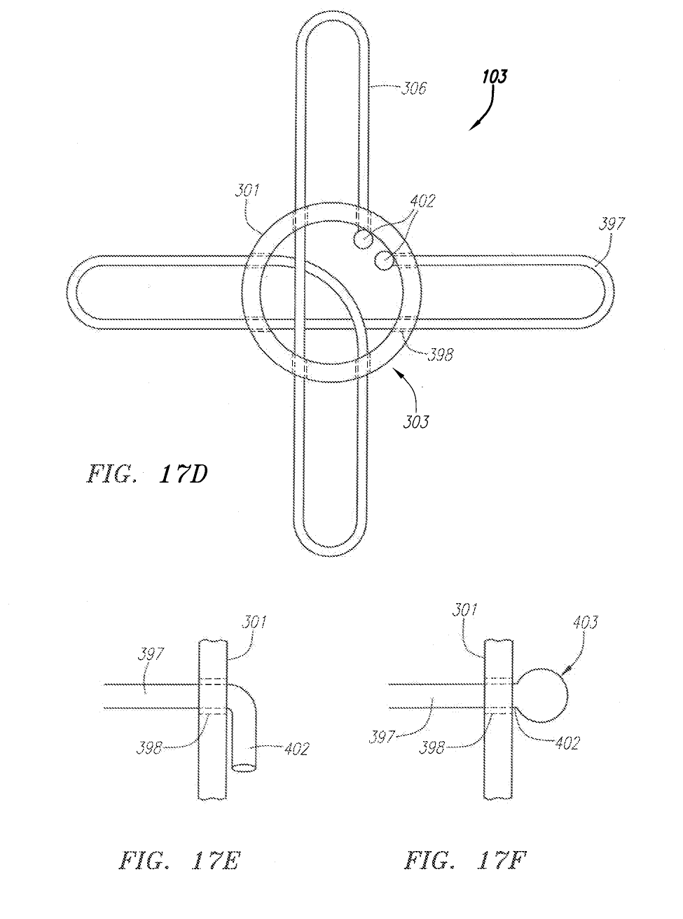

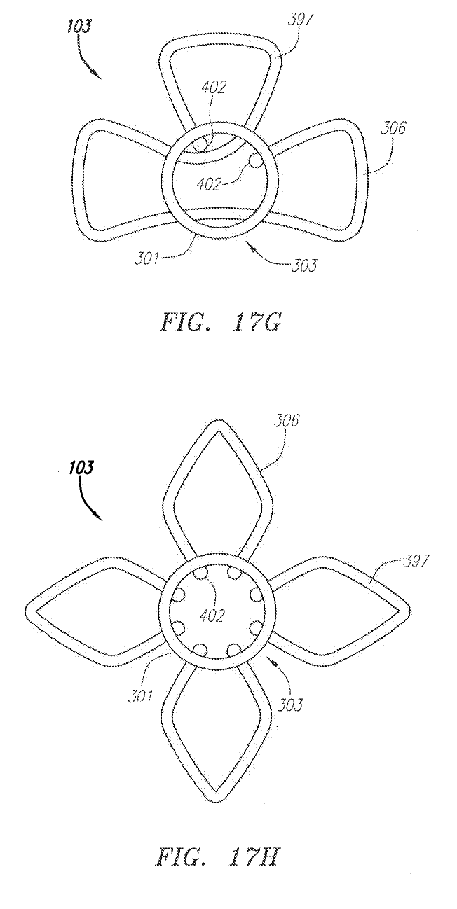

FIGS. 17A-J depict additional exemplary embodiments of clip 103 where LA and RA members 306 and 307 are formed from a separate body or bodies 397. In the perspective view of FIG. 17A, end portions 303 and 304 each include multiple apertures 398 and 399, respectively, through which a deflectable, wire-like body 397 can be routed. FIG. 17A depicts clip 103 with LA and RA members 306 and 307 in the deployed configuration. Wire-like body 397 is preferably fabricated from a superelastic material, such as NITINOL and, or an elastic material, such as stainless steel and the like, and biased to deflect towards the deployed configuration depicted here. FIGS. 17B-C are enlarged perspective views depicting end portion 303 of another exemplary embodiment of clip 103 in the undeployed configuration. These figures show that LA members 306 can be configured to deflect from the undeployed to the deployed configuration in a direction towards central portion 305 (as indicated by arrows 313 in FIG. 17B) or in a direction away from central portion 305 (as indicated by arrows 313 in FIG. 17C).

FIG. 17D is an end-on view of this embodiment of clip 103 in the deployed configuration. This figure depicts one exemplary manner of coupling wire-like body 397 with body 301. Here, wire-like body 397 is looped through each of apertures 398 (shown to be obscured with dashed lines). Wire-like body 397 has two end tips 402 which are configured to resist being pulled through apertures 398. FIGS. 17E-F are enlarged perspective views of one end tip 402 coupled with clip body 301. In the exemplary embodiment of FIG. 17E, end tip 402 is bent to a substantially ninety degree angle to resist pull-through. In the exemplary embodiment of FIG. 17F, end tip 402 has enlarged portions 403 that are larger than aperture 498 and therefore prevent pull-through. Enlarged portions 403 can formed in any manner such as by adding a solder ball, laser welding a ball shape, crimping on a radio opaque marker and the like. Although one wire-like body 397 is used to form four LA members 306 in these embodiments, it should be noted that each LA member 306 can be formed from a separate wire-like body 397.

Like the embodiments described above with respect to FIGS. 3A-16F, LA and RA members 306 and 307 can be configured in any manner desired even though fabricated from a separate body 397. For instance, LA members 306 can be arranged symmetrically or asymmetrically, as depicted in the end-on view of FIG. 17G. Also, each LA member can have any shape desired, including the polygonal shape with rounded corners depicted in the end-on view of FIG. 17H. Here, each LA member 306 is optionally formed from a separate body 397. Furthermore, body 397 is not limited to wire-like shapes and including, but not limited to ribbon-like, flap-like, petal-like, and tubular. The width and thickness of body 397 can also be varied as desired.

In other exemplary embodiments, one or more wire-like bodies 397 are used to form the entire clip 103. FIG. 171 is a frontal view depicting clip 103 with LA and RA members 306 and 307, end portions 303 and 304 and central portion 305 all formed from a single wire-like body 397. Wire-like body 397 is coiled to form central portion 305 and then shaped to form end portions 303 and 304 as well as LA and RA members 306 and 307. Wire-like body 397 in end portions 303 and 304 can have the same or a different thickness as body 397 in central portion 305. The thickness can be varied in any manner such as through grinding, electro-polishing and the like. Here, wire body 397 is looped to form end portions 303 and 304 and LA and RA members 306 and 307 and then joined to itself at junctions 406. In another embodiment, end portions 303 and 304, as well as LA and RA members 306 and 307 are formed from another wire-like body 397 that is mechanically joined (e.g., welded, soldered, crimped, glued, etc.) with the wire-like body forming central portion 305.

FIG. 17J is a frontal view depicting another exemplary embodiment of clip 103. Here, end portions 303 and 304 are each formed from tubular bodies 301-1 and 301-2, while central portion 305, LA members 306 and RA members 307 are formed from wire-like body 397. Wire-like body 397 is coiled to form central portion 305 and then routed through apertures 398 and 399 located in end portions 303 and 304 to form LA and RA members 306 and 307, respectively. Based on the description herein, one of ordinary skill in the art will readily recognize that clip 103 can be fabricated from any number of bodies 301 and 397 coupled together in any manner desired, and that clip 103 is not limited to the embodiments described with respect to FIGS. 17A-J.

The following discussion and FIGS. 18A-24D further describe the many different variations in which central portion 305 of body 301 can be configured. For instance, in the embodiments described with respect to FIGS. 3A-6C, central portion 303 is configured as a spring-like or coil-like body portion as one way to provide compressibility to allow the application of an adequate closure force to septal wall 207. FIG. 18A is a frontal view depicting an exemplary embodiment of clip 103 in having a spring-like compressive central portion 305 with multiple coiled segments 332. Central portion 305 is preferably biased to a fully compressed state as depicted here. The distance between distal end 309 and proximal end 310 in the fully compressed state is shown as distance 350. FIG. 18B is a frontal view depicting this embodiment in an expanded state. Preferably, the thickness of septal wall 207 is greater than distance 350 in order to allow clip 103 to apply an adequate closure force to septal wall 207.

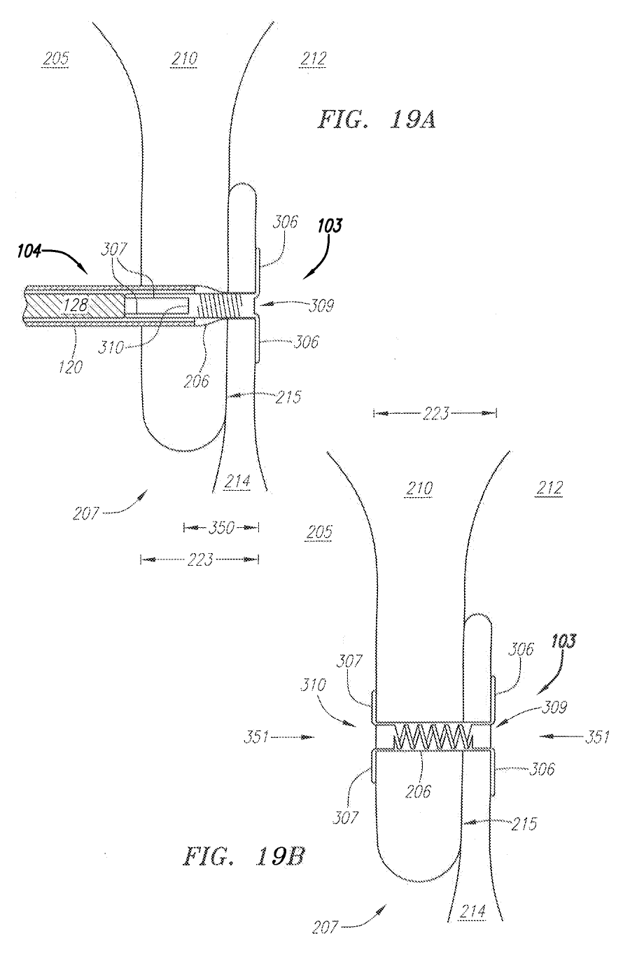

FIG. 19A is a partial cross-sectional view depicting this embodiment of clip 103 during deployment into septal wall 207. Here, septal wall 207 has a thickness 223 greater than distance 350 between distal end 309 and proximal end 310 while clip 103 is in the fully compressed state. In this embodiment, clip 103 remains in the fully compressed state until deployment of RA members 307. The deployment of RA members 307 acts to pull against LA members 306 and expand central portion 305 to accommodate the thicker septal wall 207 as depicted in FIG. 19B. The bias of central portion 305 resists this expansion and causes LA members 306 and RA members 307 to pull towards each other in directions 351. This compressive force preferably closes any PFO tunnel gap located between septum primum 214 and septum secundum 210.

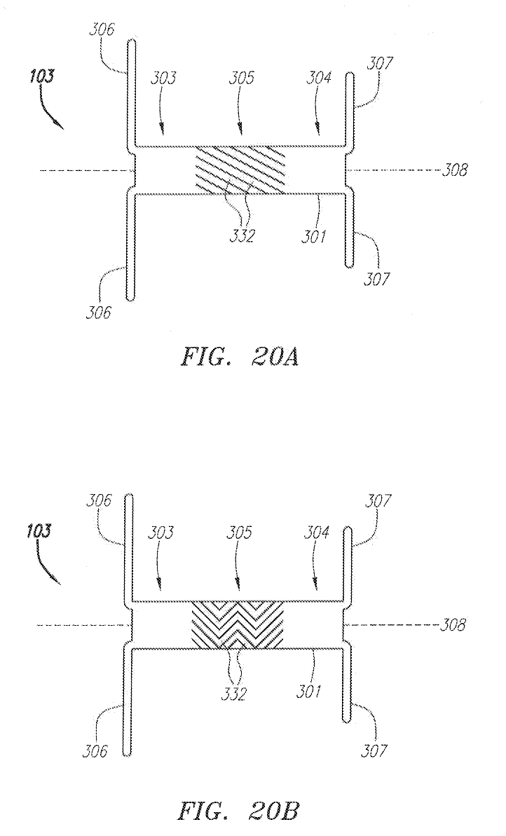

FIGS. 20A-B are frontal views depicting additional exemplary embodiments of clip 103 having various configurations of central portion 305. In FIG. 20A, central portion 305 includes a plurality of relatively straight, parallel compressive segments 332 oriented in a non-parallel manner with respect to main axis 308. In FIG. 20B, central portion 305 includes a plurality of compressive segments 332 that extend back and forth in a “zig-zag” fashion, similar to that described with respect to FIG. 71. Each of these embodiments enable central portion 305 to compress and extend in a manner similar to the spring-like embodiments described above, e.g., with respect to FIGS. 18A-B. Any desired shape for compressive segments 332 can be formed in central portion 305. In one exemplary embodiment, compressive segments 332 are formed into the desired shapes through laser cutting slots into body 301.

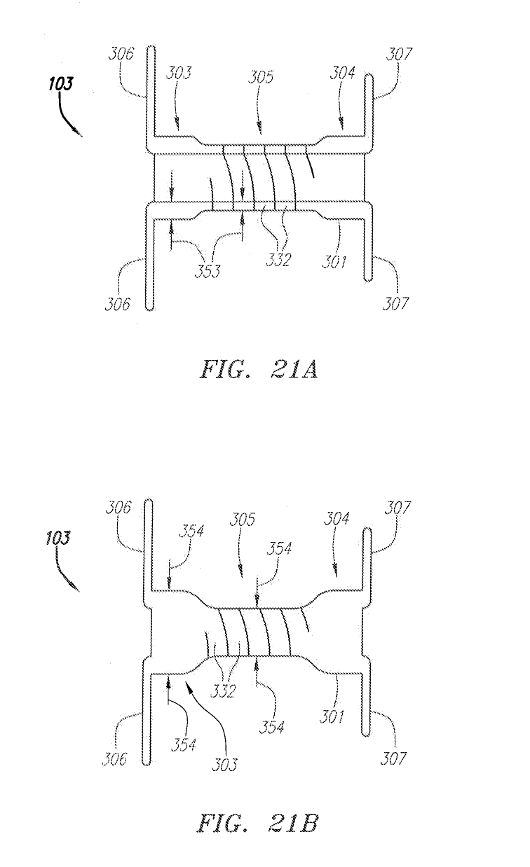

The thickness of body 301 can be varied to adjust the compliance of compressible central portion 305. FIG. 21A is a cross-sectional view depicting an exemplary embodiment of clip 103. Here, the thickness 353 of body 301 is relatively less in central portion 305 than in end portions 303 and 304. Relatively thicker end portions 303 and 304 provide enough rigidity to adequately support members 306 and 307, while a relatively thinner central portion 305 increases the compliance of the central portion 305. Any desired fabrication method can be used to adjust the thickness of body 301 including, but not limited to electro-polishing, photo-chemical etching and centerless grinding (usually relied upon to change the outer diameters).

In FIG. 21A, each coiled segment 332 has a rectangular cross-sectional shape. However, each coiled segment 332 is not limited to a rectangular shape and can be any desired shape including, but not limited to polygonal, square, circular, elliptical, irregular, symmetric, asymmetric, annular, hollow, polygonal with rounded edges, combinations thereof and the like.

In addition to the thickness of body 301, the diameter of central portion 305 can also be varied as desired. FIG. 21B is a frontal view depicting another exemplary embodiment of clip 103. Here, diameter 354 of body 301 in central portion 305 is relatively less than in end portions 303 and 304 to decrease the amount of compliance and decrease the risk of blood shunting through inner lumen 302.

It should be noted that when configured as a spring or a spring-like equivalent, central portion 305 will have an associated spring constant. This constant can be varied as desired to adjust the compression and expansion characteristics of central portion 305. The spring constant can be adjusted by varying body thickness 353, diameter 354 of central portion 305, the cross-sectional shape of compressive segments 332, the pitch between compressive segments 332, combinations thereof and the like.

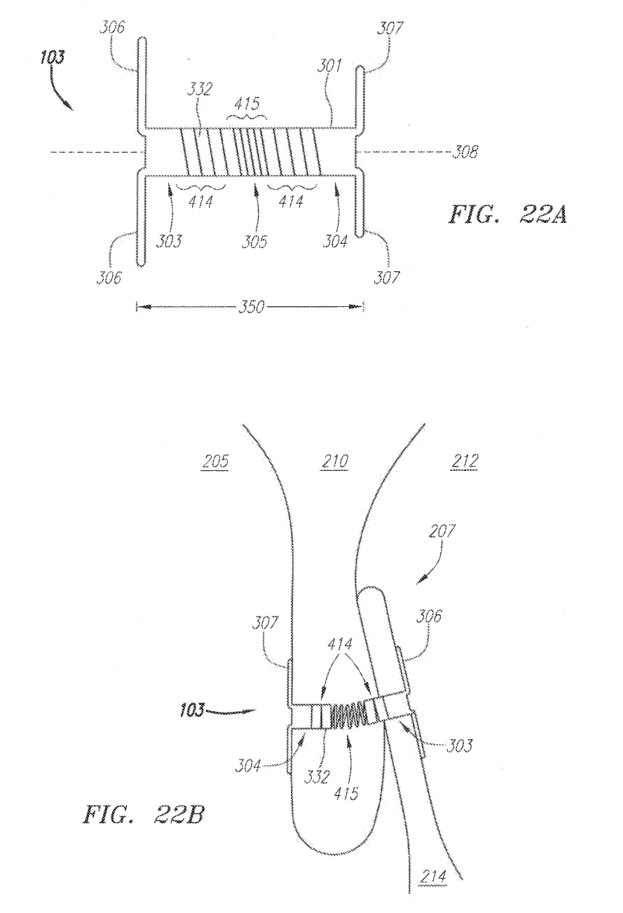

FIG. 22A is a frontal view depicting another exemplary embodiment of clip 103 where compressive segments 332 throughout central portion 305 have a varying pitch. Here, compressive segments 332 in a central region 415 of portion 305 have a relatively small pitch, while compressive segments 332 in the adjacent end regions 414 have a relatively large pitch. Generally, a smaller pitch will result in more compliance, allowing clip 103 to be expanded to a greater degree along main axis 308 and allowing clip 103 to conform to septal wall 207.

For instance, FIG. 22B is a partial cross-sectional view depicting this embodiment of clip 103 within septal wall 207. The smaller pitch of central region 415 facilitates the ability of clip 103 to bend and conform to pressure exerted by septal wall 207. The smaller pitch region 415 can also allow additional expansion of clip 103 if septal wall 207 is thick and can minimize the risk of fracture if the expansion of central portion 305 is great. It should be noted that the pitch of compressive segments 332 can be varied in any manner desired. For instance, the division of central portion 305 into multiple regions 414 and 415 is used simply to illustrate one manner in which the pitch can be varied. The pitch of segments 332 in regions 414 and 415 can be switched or the pitch can be varied in other ways not corresponding to regions 414 and 415.

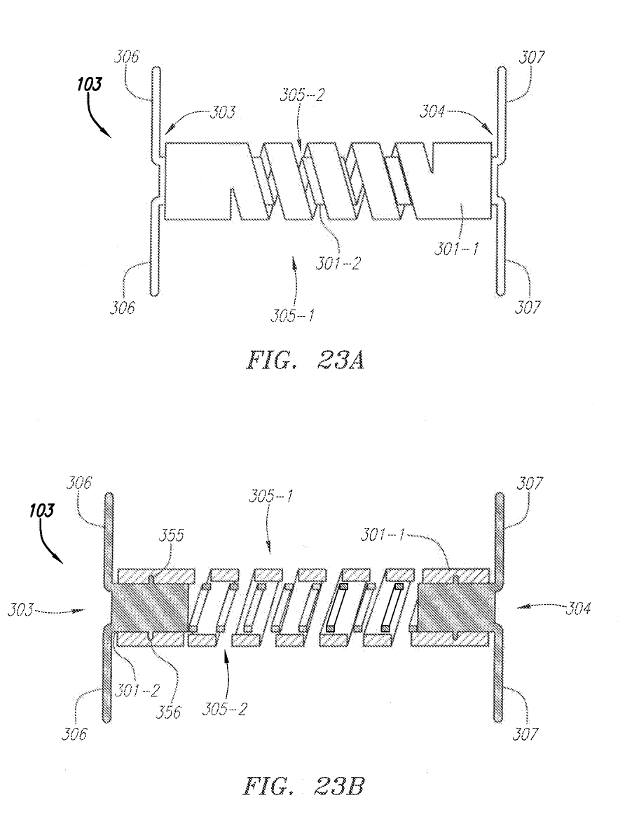

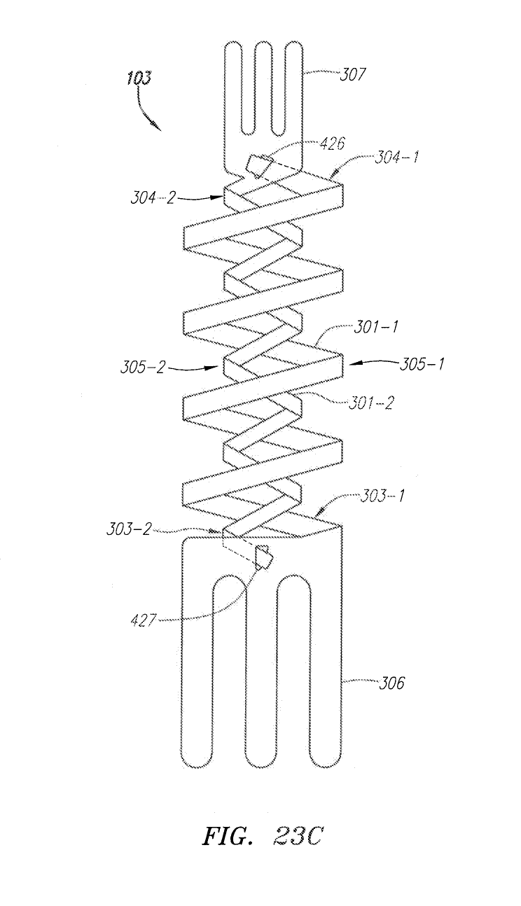

FIG. 23A is a frontal view of another exemplary embodiment of clip 103. Here, clip 103 has multiple bodies 301-1 and 302-2. In this embodiment, each body 301-1 and 301-2 includes a compressive central portion 305-1 and 305-2, respectively, the combination of which allows for the application of greater compressive forces than that of a single body 301. Outer body 301-1 has a tubular configuration and can be located around the circumference of inner body 301-2. FIG. 23B is a cross-sectional view of this embodiment of clip 103 and shows both bodies 301-1 and 301-2 in greater detail Inner body 301-2 includes a plurality of abutments 355 configured to interface with corresponding apertures 356 located in outer body 301-1. These abutments 355 act to transfer the compressive force applied by outer body 301-1 to LA and RA members 306 and 307, which are located on inner body 301-2. Any number of LA and RA members 306 and 307 can be located on inner body 301-2. Central portions 305-1 and 305-2 of bodies 301-1 and 301-2 can be configured in any manner desired to apply the desired compressive force and spring constant etc. Although not shown here, outer body 301-1 can also include one or more LA and RA members 306 and 307, in which case inner body 301-2 can have no members 306 and 307.

FIG. 23C is a frontal view depicting another exemplary embodiment of clip 103 with multiple bodies 301-1 and 301-2. In this embodiment, LA members 306 are integrally formed with body 301-1 and RA members 307 are integrally formed with body 301-2. Each body 301-1 and 301-2 includes a coiled central portion 305-1 and 305-2, respectively. Here, coiled central portion 305-1 is relatively wider than coiled central portion 301-2, and central portion 301-2 is located within the inner open region of central portion 301-1. End portion 304-1 of body 301-1 is coupled with end portion 304-2 of body 301-2, in this embodiment by routing end portion 304-1 into an aperture 426 in end portion 304-2. Likewise, end portion 303-2 of body 301-2 is coupled with end portion 303-1 of body 301-1, in this embodiment by routing end portion 303-2 into an aperture 427 in end portion 303-1. Here, each body 301-1 and 301-2 can be fabricated from a separate sheet or tubular material, heat treated under similar or different conditions and otherwise configured as desired.

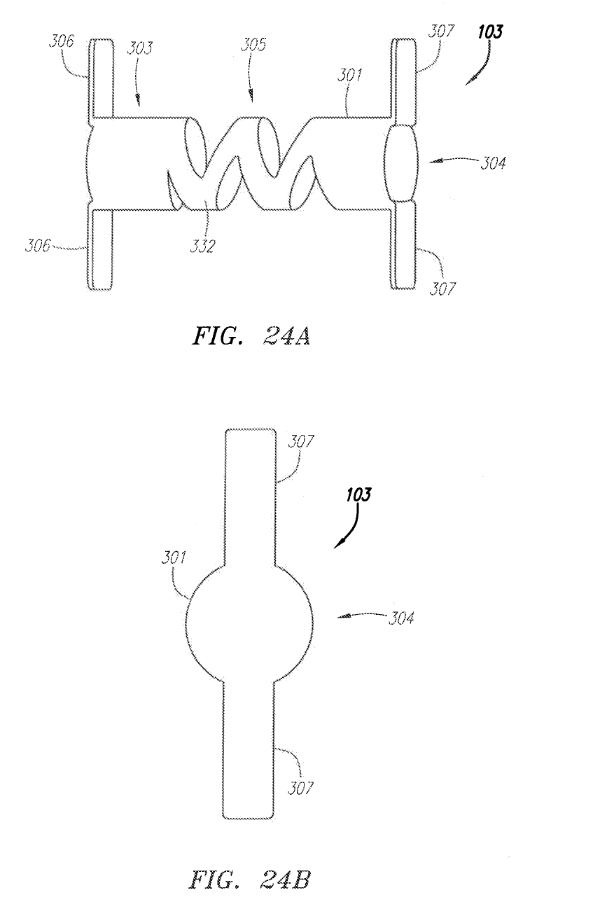

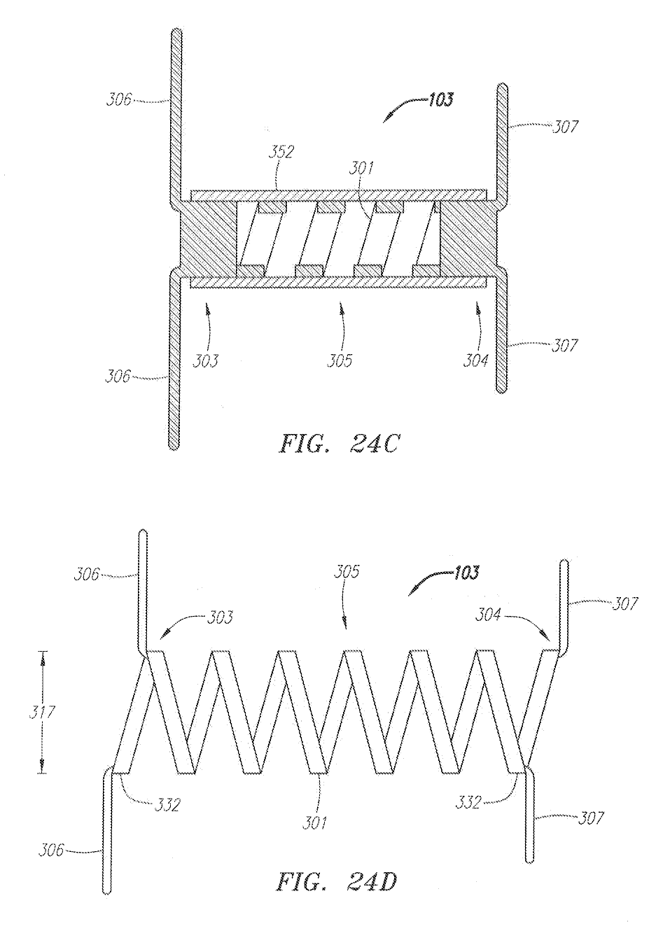

FIGS. 24A-B are frontal view and end-on views, respectively, depicting another exemplary embodiment of clip 103. In this embodiment, clip 103 has been fabricated from a solid NITINOL rod-like or cylindrical core and lacks an inner lumen. Central portion 305 is configured with multiple compressive segments 332 oriented in a symmetrical, back-and-forth “zig-zag” type fashion. This embodiment of clip 103 does not have inner lumen 302, so there is no risk of blood shunting through clip 103.

It should be noted that central portion 305 can be configured in numerous ways—only a few of which are described herein. For instance, central portion 305 can be a solid elastomeric core or can include elastomeric portions. Examples of elastomeric materials include silicone, polyurethane, polyether block amides, C-FLEX and the like. FIG. 24C is a cross-sectional view depicting an exemplary embodiment of clip 103 with an elastomeric tubular portion 352 located around the outside of coiled central portion 305 of body 301. Elastomeric tubular portion 352 can be attached to end portions 303 and 304 in any manner desired, including, but not limited to, the use of adhesives and the like. One exemplary application for elastomeric tubular portion 352 is to provide additional compressive force between end portions 303 and 304. In another exemplary embodiment, elastomeric portion 352 can encase coiled central portion 305, either partially or completely. Tubular portion 352 can also be composed of other materials such as NITINOL and stainless steel, which are not necessarily compressive in nature, and can be used to guard the inner central portion 305.

In addition, end portions 303 and 304 can also be configured to be compressible and/or expandable, such as in the exemplary embodiment depicted in FIG. 24D. Here, each of portions 303-305 are coiled and LA and RA members 306 and 307 are coupled directly to a coiled segment 332. Because end portions 303 and 304 are not solid tubular portions, width 317 (or diameter) of clip 103 can be adjusted through compression and/or expansion of clip 103.

Central portion 305 is also not required to be compressible and expandable and can be entirely rigid. Furthermore, it should be noted that each of the embodiments described with respect to FIGS. 18A-24D typically illustrate the modification of one or more characteristics of central portion 305 (e.g., length, diameter, etc.), however any or all such characteristics can be varied, modified or adjusted in any one implementation of clip 103.

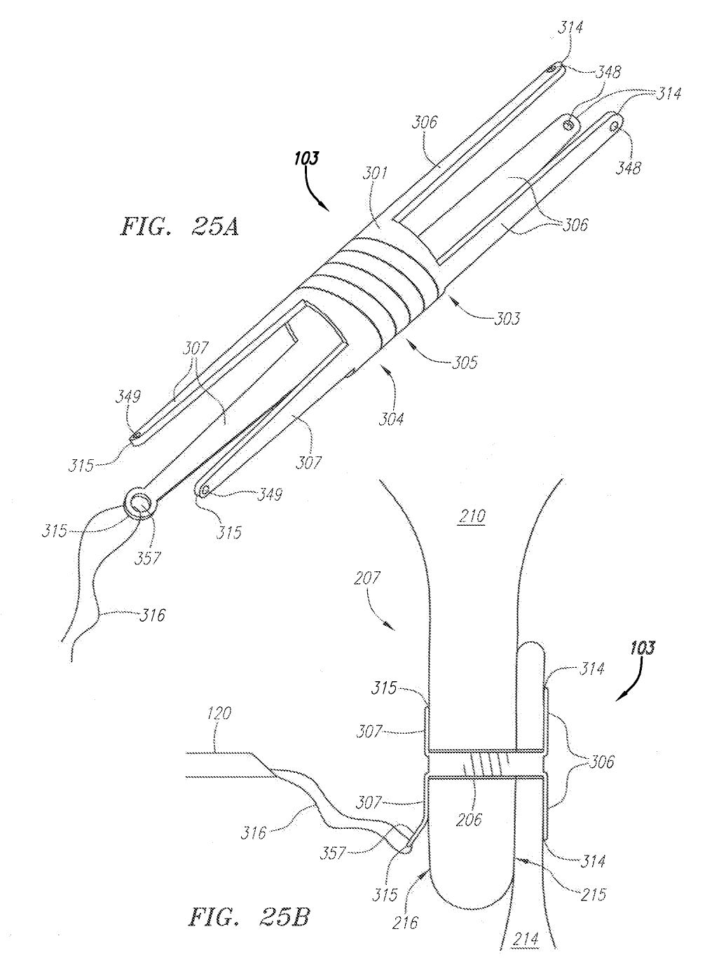

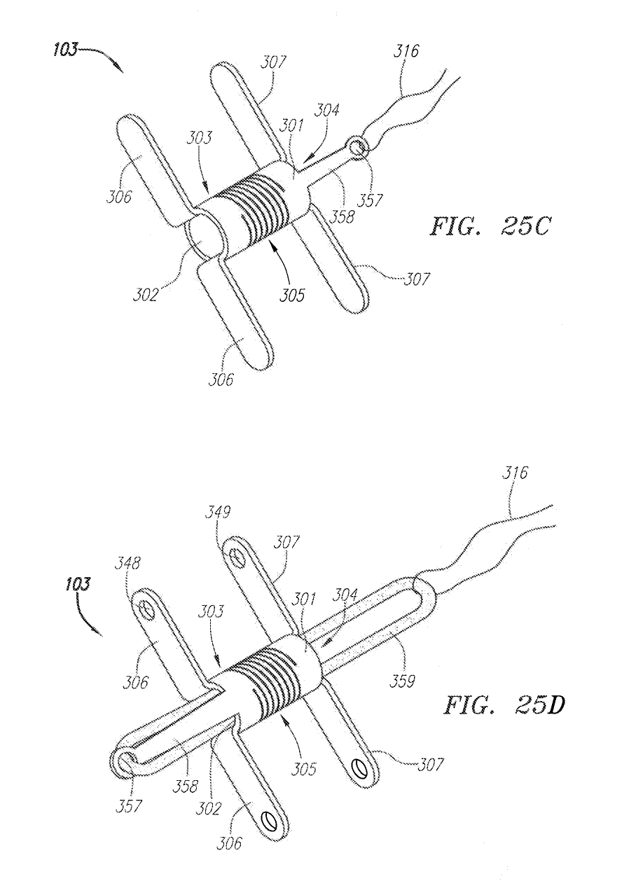

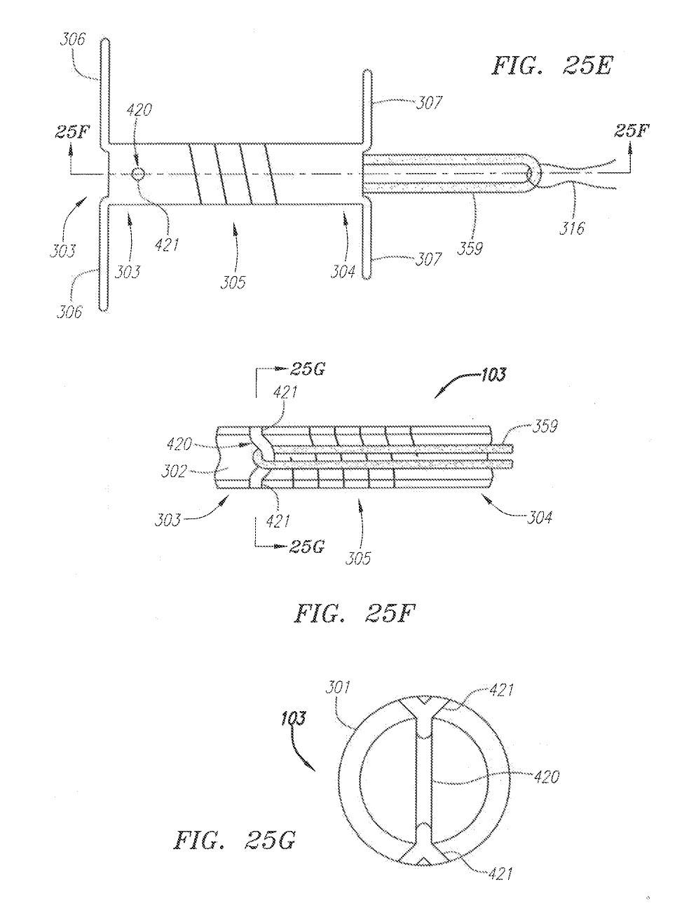

As mentioned above, retrieval tether 316 can be used to aid in removal of clip 103 if removal should become necessary during the delivery procedure. For instance, retrieval may become desirable if clip 103 is improperly deployed within septal wall 207, does not enter opening 206 and becomes free within the heart or passes through septal wall 207 into the opposing atrial chamber, etc. Retrieval tether 316 can be passed through one or more of the inner apertures 348 and 349 of end tips 314 and 315, respectively, or an additional retrieval aperture 357 can be included. FIG. 25A is a perspective view depicting another exemplary embodiment of clip 103 in the deployed configuration. Here, one RA member 307 is configured with retrieval aperture 357 located on end tip 315. Retrieval aperture 357 is relatively larger than apertures 348 and 349 to allow easier passage of tether 316. FIG. 25B is a partial cross-sectional view of this embodiment of clip 103 implanted within septal wall 207. In this embodiment, end tip 315 having retrieval aperture 357 is preferably deflected away from septum secundum surface 216 to allow for easier passage of tether 316 therethrough.

FIG. 25C is a perspective view depicting yet another exemplary embodiment of clip 103 in the deployed configuration. Here, a retrieval member 358 having retrieval aperture 357 is coupled to end portion 304 in addition to RA members 307. Retrieval member 358 is not configured to deflect and remains oriented along main axis 308. Because retrieval member 358 will extend into the blood flow path, it is relatively shorter than RA members 307. In another exemplary embodiment (not shown), retrieval aperture is formed directly in end portion 304.

In additional exemplary embodiments of clip 103, retrieval member 358 can be placed on the opposite side of clip 103 and coupled with end portion 303. In these instances, tether 316 can be routed through retrieval aperture 357 and inner lumen 302 past end portion 304 and back to delivery device 104. Tether 316 could also be additionally routed through one or more apertures 348 and 349 in LA members 306 and 307, respectively.

FIG. 25D is a perspective view depicting another exemplary embodiment of clip 103. In this embodiment, a suture 359 is looped through retrieval aperture 357 located on the LA side of clip 103 and routed through inner lumen 302. Retrieval tether 316 is looped with suture 359 and used to pull suture 359 during the retrieval process. Suture 359 can be any type of suture including, but not limited to, braided and unbraided sutures, polyester sutures, polypropylene monofilament sutures, coated sutures (e.g., flourocoated sutures and the like) bio-degradable sutures and the like. Here, suture 359 is looped, but the ends of suture 359 can also be tied or otherwise coupled through retrieval aperture 357 or any other portion of clip 103. The use of suture 359 routed through inner lumen 302 at least partially blocks inner lumen 302 and reduces the risk of blood shunting. Attachment of suture 359 (or tether 316) to LA end portion 303 also guards against the risk of clip 103 fracturing due to the mechanical stress that can be placed on central portion 305 during the retrieval process.

FIG. 25E is a frontal view depicting another exemplary embodiment of clip 103 configured for retrieval with either suture 359, tether 316 or both. FIG. 25F is a partial cross-sectional view of this embodiment taken along line 25F-25F of FIG. 25E. In this embodiment, suture 359 is wrapped around a rod-like member 420, which is coupled with clip 103 and lies across inner lumen 302. Here, member 420 is fixed within apertures 421 in body 301. Member 420 can be used for retrieval of clip 103 in place of retrieval member 358. Member 420 can be fabricated from a radio opaque material, such as tantalum, gold, platinum and the like, in order to increase the visibility of clip 103 to X-ray imaging devices.

Member 420 is shown as being bent inside lumen 302 so that it is held in place within apertures 421. However, member 420 can be coupled with body 301 in any manner desired such as with crimping, adhesives, welding and the like. Also, member 420 can be held in place with flared ends, as depicted in the cross-sectional view of FIG. 25G, which is taken along line 25G-25G of FIG. 25F.

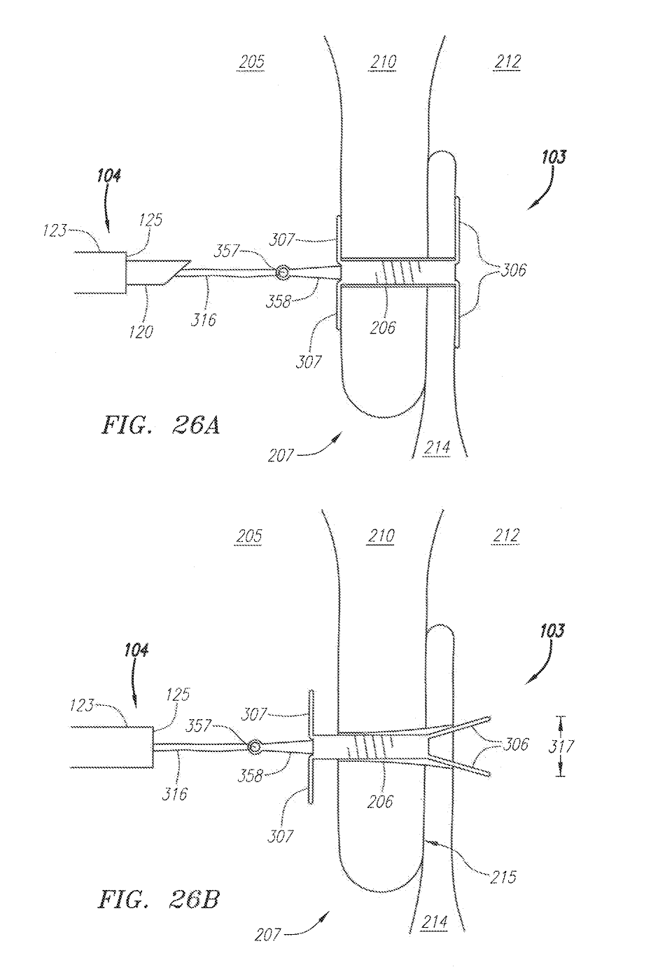

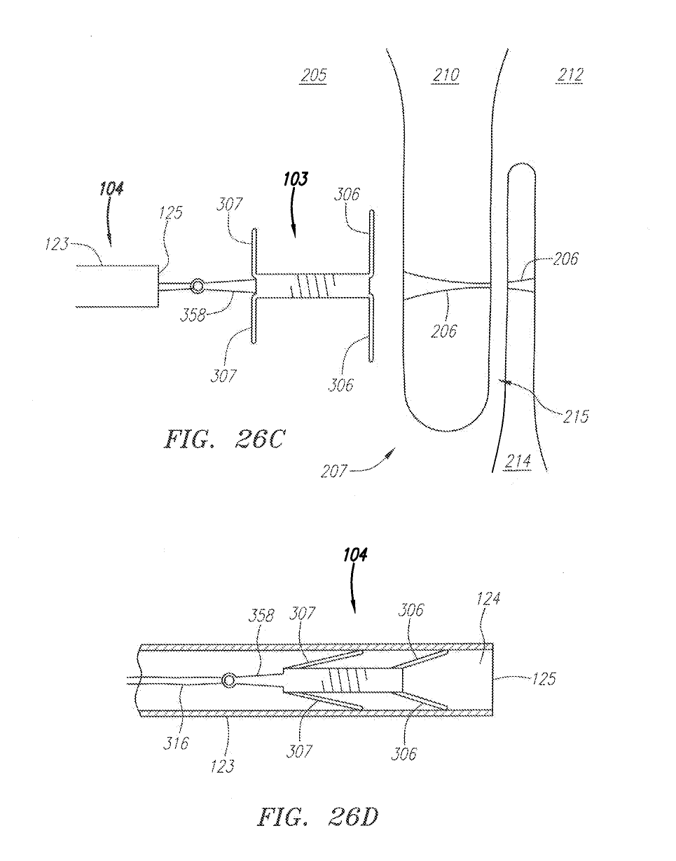

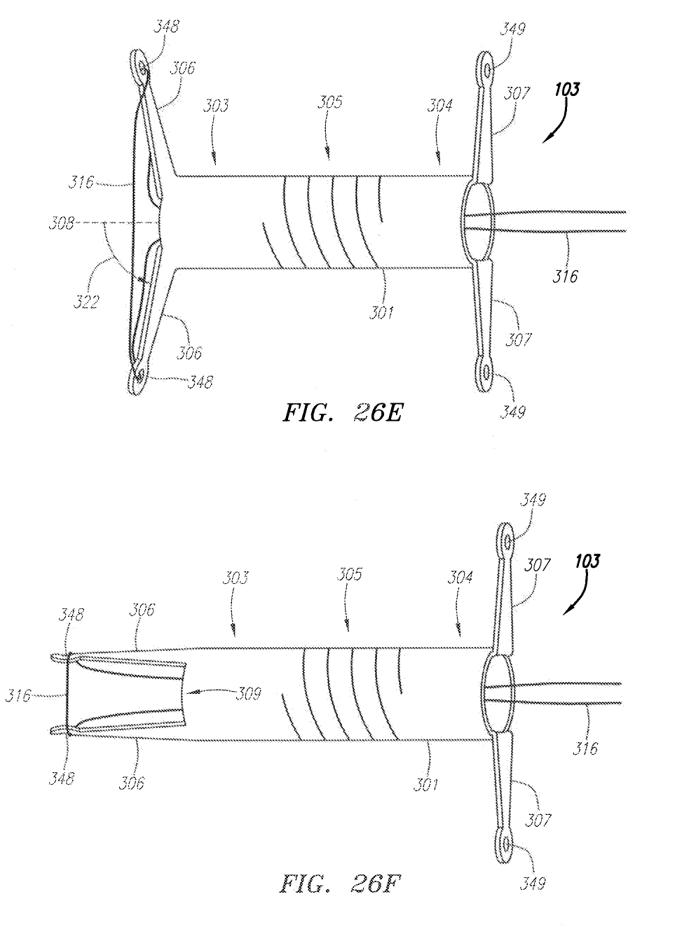

FIGS. 26A-D are partial cross-sectional views depicting one exemplary embodiment of retrieval of clip 103 after full deployment in septal wall 207. FIG. 26A depicts clip 103 deployed within septal wall 207 and tether 316 routed through retrieval aperture 357, which is located on member 358 coupled to end portion 304. In this embodiment, needle 120 is slidably disposed within outer sheath 123. In FIG. 26B, needle 120 has been retracted proximally into outer sheath 123 to prepare for retrieval of clip 103. To retrieve clip 103, tether 316 is pulled proximally, which causes clip 103 to be pulled back through opening 206 as depicted here. The force applied against LA members 306 causes members 306 to deflect back towards the undeployed configuration. This reduces the lateral cross-sectional width 317 of clip 103 and allows clip 103 to pass through opening 206.

FIG. 26C depicts clip 103 located entirely within right atrium 205 after having been pulled back through septal opening 206. Preferably, clip 103 is withdrawn into inner lumen 124 of outer sheath 123, although clip 103 can be withdrawn into any other tubular member that is or is not part of system 100. For instance, in another embodiment, another tubular member is advanced over sheath 123 and used to retrieve clip 103.

Tether 316 is continually pulled until clip 103 is brought back within lumen 124 as depicted in FIG. 26D. As can be seen, LA members 306 are deflected back into the undeployed configuration and RA members 307 are deflected away from the undeployed configuration into a new, retrieved configuration where each RA member 307 has generally the same orientation as LA members 306. Distal end 125 of outer sheath 123 can be made rigid and can be made lubricous in order to facilitate passage of clip 103 therethrough.

Tether 316 (or suture 359) can also be used to deflect LA members 306 or RA members 307 prior to retrieval. FIG. 26E is a perspective view depicting another exemplary embodiment of clip 103 in the deployed configuration where tether 316 is routed through apertures 348 in each of LA members 306 and inner lumen 302. In this embodiment, each of LA members is generally straight and has a deflection angle 322 that is less than ninety degrees to place apertures 348 in a position distal to distal end 309. With apertures 348 positioned in that manner, tension placed on tether 316 in a proximal direction will cause LA members 306 to deflect distally back towards the undeflected, pre-deployment configuration as shown in FIG. 26F. Use of tether 316 to retrieve clip 103 can therefore also cause LA members 306 to deflect into a position less likely to damage septal wall 207 while being pulled back through opening 206.

During deployment of clip 103, tether 316 can also be used to control the deployment of LA members 306 or RA members 307. FIG. 26G is a frontal view depicting additional an exemplary embodiment of clip 103 during deployment (septal wall 207 is not shown). Here, clip 103 includes three RA members 307-1, 307-2 and 307-3. Tether 316 is routed through two apertures 425 in RA end portion 304 and also through aperture 349-1 in RA member 307-1. Tension is maintained on tether 316, which keeps RA member 307-1 in the undeployed configuration while RA members 307-2 and 307-3 are left free to deflect, as depicted in FIG. 26G. This can facilitate orientational adjustment of clip 103. Once clip 103 is oriented as desired, the remaining RA member 307-1 can be allowed to deflect by loosening tether 316. In this manner, control of the order of deployment of RA members 307 can be accomplished. Of course, additional members can be controlled with tether 316 as desired.

When proper implantation of clip 103 is achieved and the need to retrieve clip 103 is eliminated, tether 316 is preferably severed and removed from clip 103. This is preferably done with a cutting device located within delivery device 104 in a manner readily apparent to those of ordinary skill in the art. Alternatively, tether 316 can be severed with heat, electricity, mechanical vibration, chemicals and the like. In one exemplary embodiment, tether 316 can be configured with a load dependent coupling configured to break when a predetermined load is applied to tether 316, thus eliminating the need for an additional cutting device.

It should be noted that these are just one set of exemplary embodiments of a retrieval structure and method and, as one of ordinary skill in the art will readily recognize, other structures and methods of retrieval are possible depending on the configuration of clip 103, the retrieval device (e.g., a tether or other device), the desired retrieved configuration and the like.

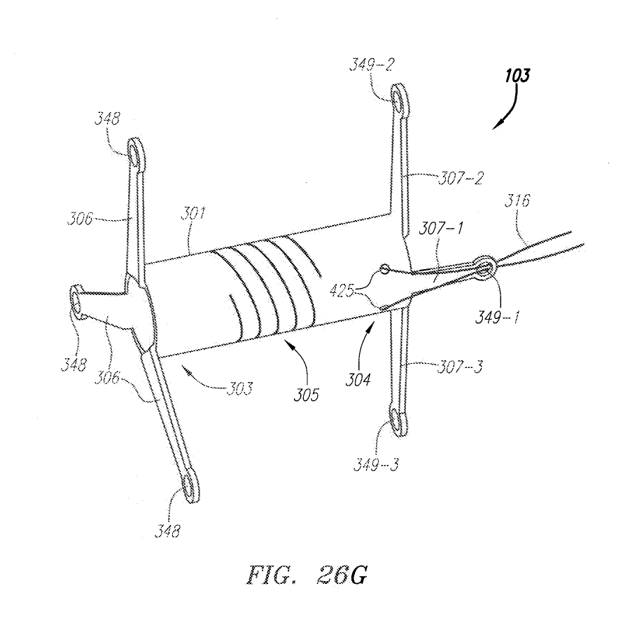

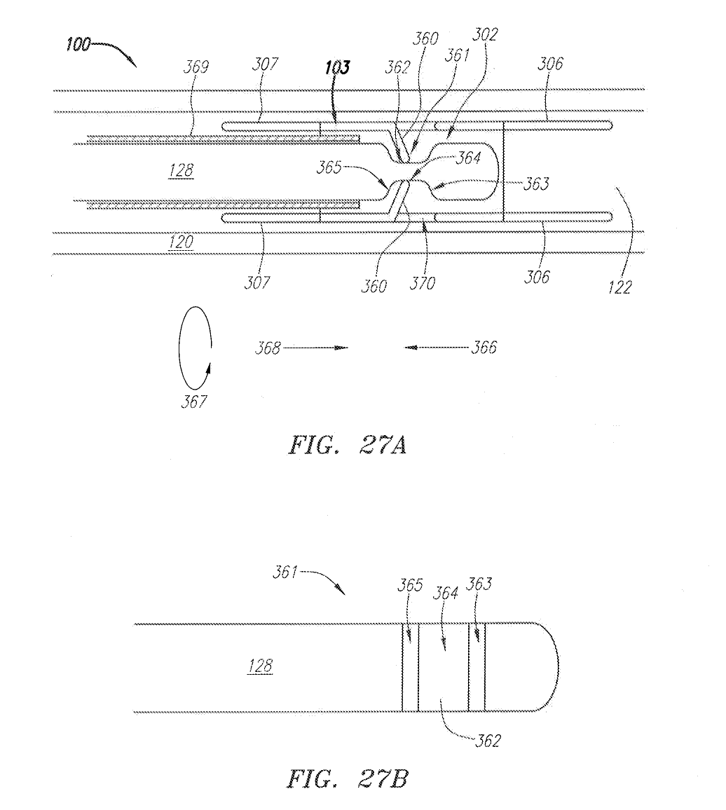

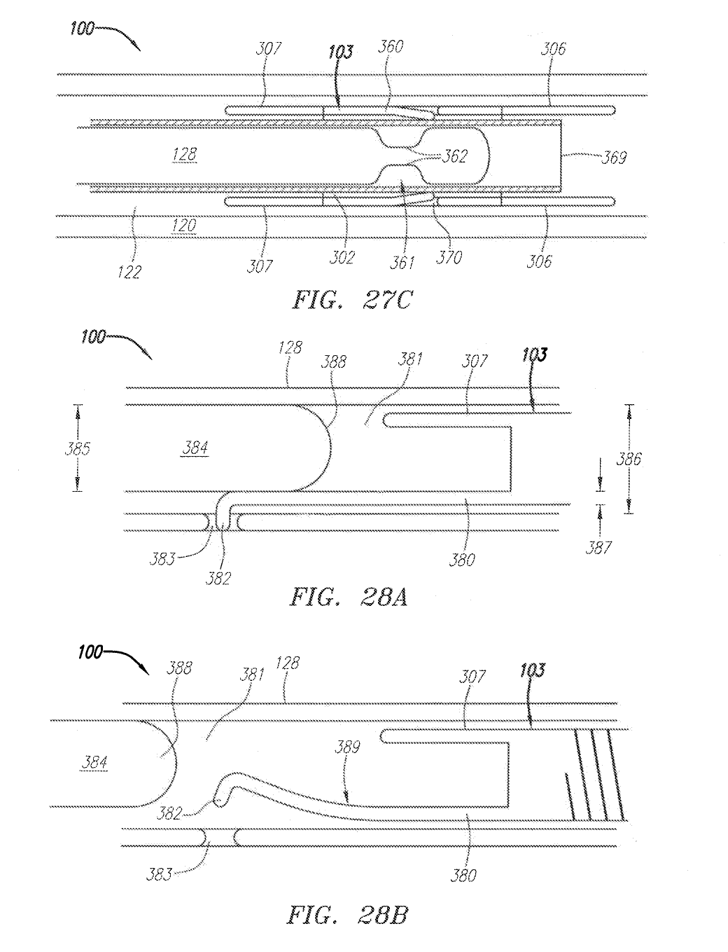

As mentioned above, it can be desirable to control the radial orientation of clip 103 during delivery. FIG. 27A is a partial cross-sectional view depicting an exemplary embodiment of treatment system 100 configured to allow adjustment of the radial orientation of clip 103. Here, clip 103 is shown within needle 120. Clip 103 has opposing inwardly deflected tabs 360, which are configured to extend into inner lumen 302. Pusher member 128 is also shown located within lumen 122 of needle 120 and inner lumen 302 of clip 103. Pusher member 128 is generally cylindrical except for a distal portion 361. In distal portion 361, pusher member 128 includes opposing indentations 362 configured to interface with tabs 360.

Indentations 362 are preferably formed with three surfaces, a distal surface 363 configured to abut tab 360 when pusher member 128 is retracted proximally in direction 366 and thereby cause clip 103 to move proximally with pusher member 128, an intermediate surface 364 configured abut tab 360 when pusher member 128 is rotated in radial direction 367 and thereby cause clip 103 to be rotated radially with pusher member 128, and a proximal surface 365 configured to abut tab 360 when pusher member 128 is advanced distally in direction 368 and thereby cause clip 103 to move with pusher member 128 when advanced distally. FIG. 27B is a lateral view of this exemplary embodiment of pusher member 128 rotated 90 degrees from the depiction in FIG. 27A. Here, indentations 362 can be seen from a different perspective.

Thus, in this embodiment, by manipulating pusher member 128, a user is capable of controlling the radial orientation of clip 103, such as to position LA members 306 and RA members 307 as desired. The user is also enabled to adjust the position of clip 103 both distally and proximally. This embodiment also provides retainment/retrieval capability to the user, as an alternative or supplement to retrieval tether 316.

An inner tubular member 369 is also shown for unlocking clip 103 from pusher member 128. Once clip 103 is properly positioned and ready to be released from pusher member 128, tubular member 369 can be advanced distally to cause tabs 360 to deflect outwards from inner lumen 302. Tabs 360 are preferably formed by cutting slots 370 into body 301, allowing tabs 360 to deflect outwards into slots 370 when forced by member 369. FIG. 27C is a another partial cross-sectional view depicting this exemplary embodiment of treatment system 100 with tubular member 369 advanced into a position to unlock clip 103.

One of ordinary skill in the art will readily recognize that various other configurations will also allow clip 103 to be controlled in distal, proximal and radial directions. For instance, tabs 360 can be located on pusher member 128 and configured to interface with indentations 362 located in clip body 301. Also, one of ordinary skill in the art will readily recognize that other locking structures, such as clamps, lock and key structures and the like, can be used in place of tabs 360 and indentations 362.

Yet another exemplary embodiment of treatment system 100 allowing both retainment/retrieval capability and orientational control of clip 103 is depicted in the partial cross-sectional view of FIG. 28A. Here, clip 103 includes an RA member 380 configured to interlock with the portion of delivery device 104 from which it is delivered, e.g., needle 120, pusher member 128 or another member of system 100. In this embodiment, pusher member 128 is configured as tubular member having an inner lumen 381 for housing a proximal portion of clip 103 including RA member 380. RA member 380 is similar to RA members 307 in that both are deflectable to abut septum secundum surface 216. Here, RA member 380 is also relatively longer than RA member 307. RA member 380 also has a curved or bent end tip 382 configured to interface with a slot 383 in pusher member 128.

A holding member 384 is preferably slidably disposed within inner lumen 381 of pusher member 128. Holding member 384 is configured to maintain RA member 380 in a position within slot 383 as depicted in FIG. 28A. In this embodiment, holding member 384 has a thickness 385 that is sized to be approximately equal to the diameter 386 of inner lumen 128 less the thickness 387 of RA member 380. When distal end 388 of holding member 384 is positioned distally past slot 383 with RA member 380 placed therein, holding member 384 forces RA member 380 to maintain in place within slot 383. Thus, clip 103 is prevented from separation from pusher member 128 while RA member 380 is maintained within slot 383. Also, any distal, proximal or rotational movement of pusher member 128 will translate to clip 103, thereby allowing control of the position and orientation of clip 103 as well as retrieval of clip 103 after deployment.

To allow clip 103 to be separated from pusher member 128, holding member 384 is preferably retracted proximally until distal end 388 is positioned proximal to slot 383, as depicted in the partial cross-sectional view of FIG. 28B. This allows RA member 380 to freely withdraw from within slot 383, thereby unlocking clip 103 from pusher member 128.

In order to facilitate withdrawal from within slot 383, RA member 380 is preferably biased to deflect to a withdrawn position as depicted in FIG. 28B. Here, RA member 380 has a curved or bent portion 389 oriented such that end tip 382 is deflected into inner lumen 381 of pusher member 128 once holding member 384 is removed. RA member 380 can be configured with portion 389 through heat treatment and the like.

In many of the embodiments described above, clip 103 has a generally cylindrical, tubular body 301. It should be noted that clip 103 is not limited to cylindrical or tubular bodies 301. For instance, the radial cross-sectional shape of body 301 can be any shape including, but not limited to, circular, elliptical and other curved shapes, triangular, square, rectangular, hexagonal and other polygonal shapes, irregular shapes, symmetrical and asymmetrical shapes, polygonal shapes with rounded corners, combinations thereof, and the like.

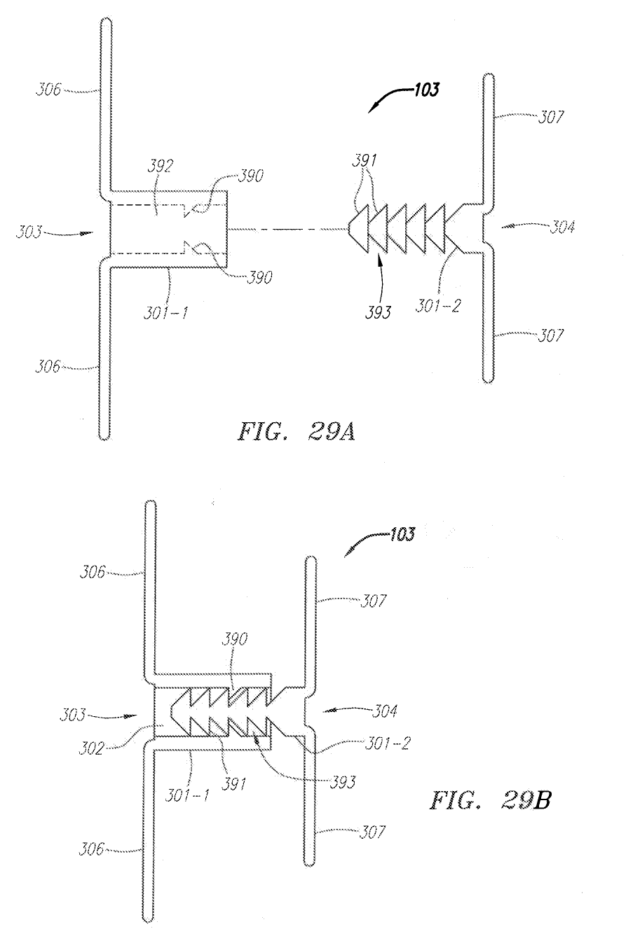

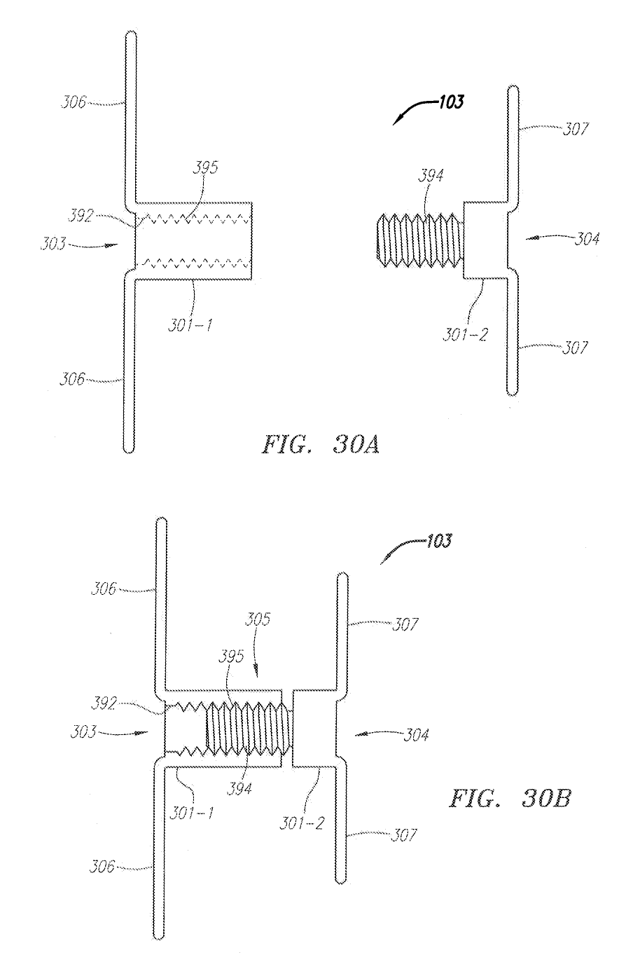

Instead of, or in addition to, compressive central portion 305, clip 103 can be configured with adjustable interlocking capability, i.e., the capability to adjust the distance between LA and RA members 306 and 307 by a desired amount and then lock that distance in place. FIGS. 29A-30B depict exemplary embodiments of clip 103 configured with incremental interlocking capabilities.

FIGS. 29A-B depict an exemplary embodiment of clip 103 having two separate bodies 301-1 and 301-2 configured to ratchet together. FIG. 29A is a frontal view of LA body 301-1 and RA body 301-2 in an uncoupled state. Here, LA body 301-1 is tubular and includes inner lumen 392 (indicated as obscured within body 301-1 by the dashed line) and LA members 306. RA body 301-2 is configured to slide within inner lumen 392 and includes RA members 307. LA body 301-1 preferably includes one or more LA abutments 390 configured to interface with corresponding RA abutments 391 included on RA body 301-2. LA abutments 390 and RA abutments 391 can be configured in any manner desired. In this exemplary embodiment, LA abutments 390 are opposing deflectable tabs formed in the tubular body 301-1 and RA abutments 391 are a series of conical outcroppings formed along the length of body 301-2.

FIG. 29B is a cross-sectional view showing clip 103 in the coupled, deployed configuration. Here, RA body 301-2 has been advanced into lumen 392 such that tabs 390 can interface with the conical abutments 391. Tabs 390 are preferably deflectable into slots 393 located between conical abutments 391. In this embodiment, tabs 390 are configured to deflect into slots 393 as RA body 301-2 is advanced into inner lumen 392. The conical shape of abutments 391 allows tabs 390 to deflect as RA body 301-2 is advanced into inner lumen 302, yet prevents RA body 301-2 from being retracted proximally out of inner lumen 302. This effectively locks bodies 301-1 and 301-2 together with septal wall 207 located therebetween. This allows the length of clip 103 to be adjusted to compensate for septal walls 207 having varying thicknesses.

It should be noted that the size of each indentation 391 can be adjusted to provide the desired number of locking positions per unit of length of clip 103. Also, clip 103 can be configured with a compressible/expandable central portion 305 if desired, in addition to the interlocking capability provided by ratcheting abutments 390-391.

FIGS. 30A-B depict another exemplary embodiment of clip 103 configured with adjustable interlocking capability. In this embodiment, LA body 301-1 and RA body 301-2 are threaded and configured to screw together. FIG. 30A is a frontal view of clip 103 in an uncoupled state depicting RA body 301-2 having threads 394, which are configured to interface with corresponding threads 395 in inner lumen 392 of LA body 301-1 (both indicated as obscured within body 301-1 by the dashed line). FIG. 30B is a cross-sectional view depicting clip 103 in a coupled state. The size of each thread 394 and 395 can be adjusted to provide the desired amount of length adjustment per amount of rotation of body 301-2 with respect to body 301-1.

It should be noted that configuration of abutments 390 and 391 and threads 394 and 395 can be switched between LA and RA bodies 301-1 and 301-2. In other words, RA body 301-2 can include inner lumen 392 and LA body 301-1 can be ratcheted or screwed into RA body 301-2.

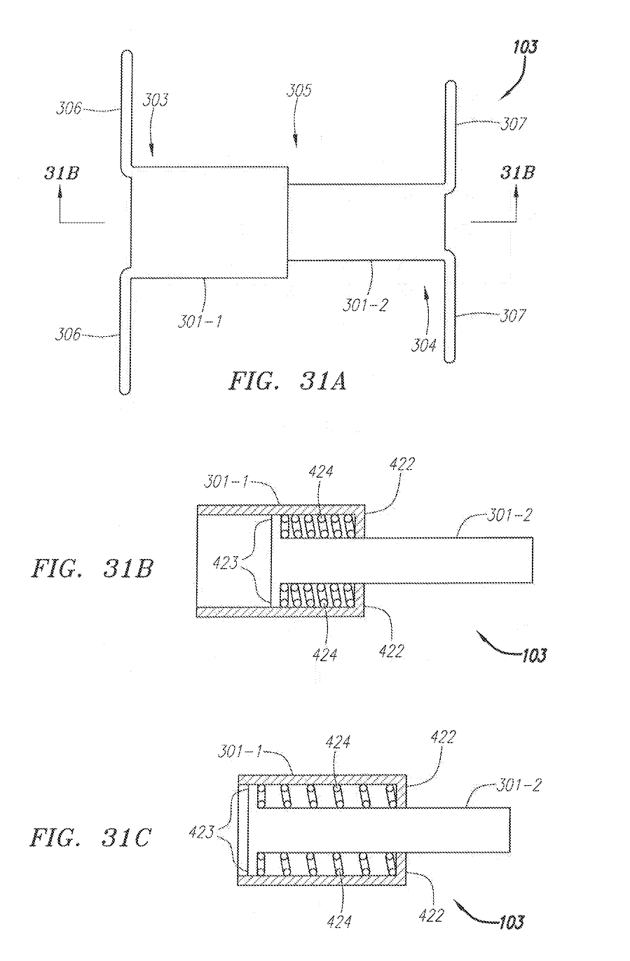

FIGS. 31A-C depict another exemplary embodiment of clip 103 having multiple bodies 301-1 and 301-2. Like many of the previous embodiments, clip 103 is configured to expand and compress as needed. FIG. 31A is a perspective view of clip 103 and FIG. 31B is a cross-sectional view of clip 103 taken along line 31B-31B of FIG. 31A. In this embodiment, body 301-1 is tubular and configured to slide over body 301-2. Body 301-1 can include one or more LA members 306 and body 301-2 can include one or more RA members 307. On the ends opposite LA and RA members 306 and 307, bodies 301-1 and 301-2 each have opposing abutments 422 and 423, respectively, which are configured to guide the movement of each body 301-1 and 301-2 and also to serve as a point against which one or more bias elements 424 can apply pressure. Here, two spring-like bias elements 424 are shown in the gap between bodies 301-1 and 301-2, although any number and type of bias elements can be used. Bias elements 424 are configured to apply expansive pressure against abutments 422 and 423 to bias clip 103 towards the fully compressed state depicted in the cross-sectional view of FIG. 31C.

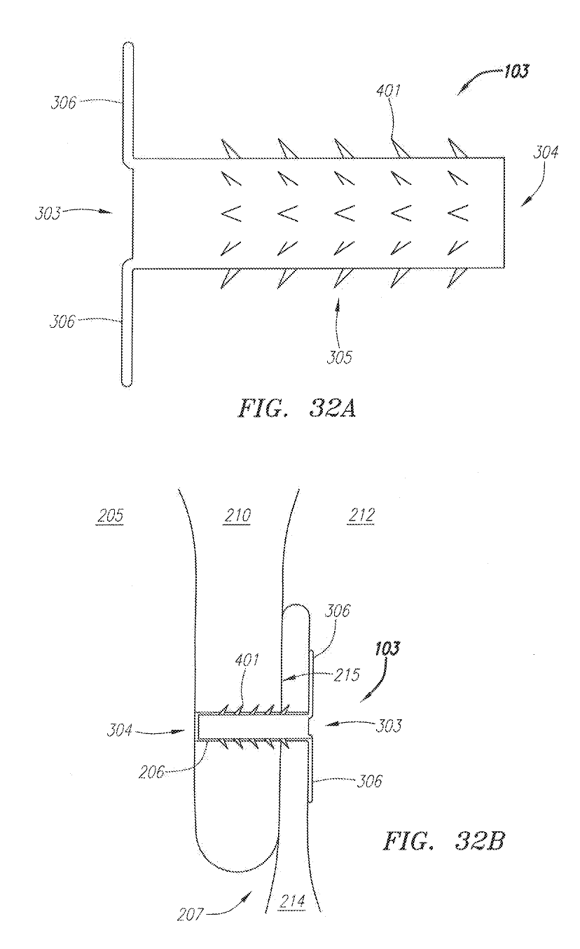

In the above embodiments described with respect to FIGS. 3A-31B, clip 103 has included both LA members 306 and RA members 307 for contacting opposing septal surfaces 213 and 216 and pulling those surfaces 213 and 216 together to preferably close any PFO tunnel 215. However, clip 103 can be configured without one or both of external members 306 and 307. For instance, FIG. 32A is a frontal view depicting an exemplary embodiment of clip 103 configured with LA members 306 only. Instead of RA members 307, clip 103 includes tines 401 configured to grasp the interior of septum secundum 210 in order to close any tunnel 215 located between septum primum 214 and septum secundum 210.

FIG. 32B is a partial cross-sectional view depicting this embodiment of clip 103 implanted within septal wall 207. Clip 103 can also be implanted through septal wall 207 adjacent to tunnel 215, if desired. In another exemplary embodiment, clip 103 includes RA members 307 and uses tines 401 in place of LA members 306. In yet another exemplary embodiment, LA members 306 and RA members 307 are omitted and only tines 401 are used to draw septum primum 214 and septum secundum 210 together. It should be noted that any type of grasping structure or abrasive surface can be used with or instead of tines 401.

It should be noted that any feature, function, method or component of any embodiment described with respect to FIGS. 1-32B can be used in combination with any other embodiment, whether or not described herein. As one of skill in the art will readily recognize, treatment system 100 and the methods for treating a septal defect can be configured or altered in an almost limitless number of ways, the many combinations and variations of which cannot be practically described herein.

The devices and methods herein may be used in any part of the body, in order to treat a variety of disease states. Of particular interest are applications within hollow organs including but not limited to the heart and blood vessels (arterial and venous), lungs and air passageways, digestive organs (esophagus, stomach, intestines, biliary tree, etc.). The devices and methods will also find use within the genitourinary tract in such areas as the bladder, urethra, ureters, and other areas.

Other locations in which and around which the subject devices and methods find use include the liver, spleen, pancreas and kidney. Any thoracic, abdominal, pelvic, or intravascular location falls within the scope of this description.

The devices and methods may also be used in any region of the body in which it is desirable to appose tissues. This may be useful for causing apposition of the skin or its layers (dermis, epidermis, etc), fascia, muscle, peritoneum, and the like. For example, the subject devices may be used after laparoscopic and/or thoracoscopic procedures to close trocar defects, thus minimizing the likelihood of subsequent hernias. Alternatively, devices that can be used to tighten or lock sutures may find use in various laparoscopic or thoracoscopic procedures where knot tying is required, such as bariatric procedures (gastric bypass and the like) and Nissen fundoplication. The subject devices and methods may also be used to close vascular access sites (either percutaneous, or cut-down). These examples are not meant to be limiting.

The devices and methods can also be used to apply various patch-like or non-patchlike implants (including but not limited to Dacron, Marlex, surgical meshes, and other synthetic and non-synthetic materials) to desired locations. For example, the subject devices may be used to apply mesh to facilitate closure of hernias during open, minimally invasive, laparoscopic, and preperitoneal surgical hernia repairs.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit of the disclosure.

What is claimed is:

1. A treatment system for treating a septal defect, comprising:
an implantable device, comprising an elongate body portion having a first end, a second end, and a variable length section located therebetween, the implantable device further comprising a first arm-like member coupled with the first end and a second arm-like member coupled with the second end, the arm-like members being configured to move from a stressed, undeployed configuration to an unstressed, deployed configuration, wherein the implantable device is configured to at least partially close a septal defect in a septal wall when the first and second arm-like members are in the deployed configuration; and
a delivery device configured to hold the undeployed arm-like members in a stressed condition prior to delivery of the implantable device, the delivery device comprising an elongate, flexible, needle-like member configured to pierce at least a portion of the septal wall.
